(19) United States
(12) Patent Application Publication
(10) Pub. No.: US 2023/0101090 A1
(43) Pub. Date: Mar. 30, 2023

(54) System and Method for Restoring Robotic Assemblies to One Or More Self-Supporting Stable Support Positions

(71) Applicant: Sarcos Corp., Salt Lake City, UT (US)

(72) Inventors: Fraser M. Smith, Salt Lake City, UT (US); Marc X. Olivier, Salt Lake City, UT (US)

(21) Appl. No.: 17/486,362

(22) Filed: Sep. 27, 2021

(51) Int. Cl. A61H 3/00; B25J 17/00; B25J 9/16

(52) U.S. Cl. CPC A61H 3/00; B25J 17/00; B25J 9/1633; A61H 2201/1671; A61H 2201/1472; A61H 2201/1659; A61H 2201/165; A61H 2201/164; A61H 2201/1628

(57) ABSTRACT

A robotic assembly comprises a first joint comprising first and second support members rotatably coupled together, and a joint position restoration assembly coupled to at least one of the first or second support members. The joint position restoration assembly can comprise a first spring and a mechanical linkage, wherein the joint position restoration assembly is operable to apply a restoring torque to the first joint. The joint position restoration assembly can be configured to provide a restoring torque versus joint position profile relative to the first joint that corresponds to known mass properties of at least a portion of the robotic assembly acting on or otherwise associated with the first joint, such that, when the first joint is not undergoing powered actuation, the joint position restoration assembly operates to apply, based on the profile, the restoring torque to position and to support the first joint in a stable support position.

### BACKGROUND

Certain robotic assemblies, such as humanoid robots, exoskeletons (those worn or not worn by an individual), can be relatively expensive and quite heavy, which may require some other system for storing the robotic assembly, such as an exoskeleton when not worn and operated by the individual (i.e., unpowered). This can involve hanging the robotic assembly from an overhead hook or other structural support. In cases where the robotic assembly is not supported and/or restrained when unpowered, the robotic assembly will adopt a pose that is hill defined. Storing a robotic assembly in such a way can result in damage to components of the robotic assembly, and can cause fatigue or injury to individuals that must lift the robotic assembly, such as to attach it to a wearer/user of in the case of an exoskeleton, or to mount it on a support structure. Alternatively, a machine, such as a forklift or powered hoist may be used to lift and hold the robotic assembly when not in use, or while being donned by an individual, as in the case of an exoskeleton.

### BRIEF DESCRIPTION OF THE DRAWINGS

FIG. 1A illustrates an exoskeleton in a position worn by a user, in accordance with one exemplary embodiment.

FIG. 1B illustrates the exoskeleton of FIG. 1A, and in a stable standing position, after the user has stepped out of the exoskeleton.

FIG. 2A illustrates an exoskeleton in a position worn by a user, in accordance with one exemplary embodiment.

FIG. 2B illustrates the exoskeleton of FIG. 2A, and in a transition position, after the user as stepped out of the exoskeleton.

FIG. 2C illustrates the exoskeleton of FIG. 2B, and in a stable standing position, after the user has stepped out of the exoskeleton.

FIG. 3A illustrates an exoskeleton in a position worn by a user, in accordance with one exemplary embodiment.

FIG. 3B illustrates the exoskeleton of FIG. 3A, and in a transition position, after the user has stepped out of the exoskeleton.

FIG. 3C illustrates the exoskeleton of FIG. 3B, and in a stable standing position, after the user has stepped out of the exoskeleton.

FIG. 4A illustrates a schematic diagram of a robotic system having an upper robotic assembly rotatably coupled to a base platform via one or more joints in series, with the robotic system having a joint position restoration assembly associated and operable with the one or more joints.

FIG. 4B illustrates a schematic diagram of a robotic system having an upper robotic assembly rotatably coupled to a base platform via one or more joints in parallel and in series, the diagram further illustrating the robotic system having a plurality of joint position restoration assemblies, each associated and operable with a respective joint of the plurality of joints in parallel, or a single joint position restoration assembly associated and operable with a plurality of joints in parallel.

FIG. 5A illustrates an exoskeleton in a position worn by a user, in accordance with one exemplary embodiment.

FIG. 5B illustrates the exoskeleton of FIG. 5A, and in a stable upright support position, after the user has stepped out of the exoskeleton.

FIG. 6A illustrates an exoskeleton in a position worn by a user, in accordance with one exemplary embodiment.

FIG. 6B illustrates the exoskeleton of FIG. 6A, and in a stable upright support position, after the user has stepped out of the exoskeleton.

FIG. 7A illustrates an exoskeleton in a position worn by a user, in accordance with one exemplary embodiment.

FIG. 7B illustrates the exoskeleton of FIG. 7A, and in a stable upright support position, after the user has stepped out of the exoskeleton.

FIG. 8A illustrates an isometric view of a right leg section of an exoskeleton, and in a stable standing position, after the user has stepped out of the right leg section of the exoskeleton, in accordance with one exemplary embodiment.

FIG. 8B illustrates rear side view of a portion of the right leg section of FIG. 8A.

FIG. 8C illustrates a close up view of the right leg section of FIG. 7A, and showing a spring support body exploded or isolated from the right leg section.

FIG. 9A is a front schematic view of a portion of a robotic assembly, illustrating a joint and a spring, in accordance with one exemplary embodiment.

FIG. 9B is a top-down schematic view of a spring usable with the joint of FIG. 9A, in accordance with one exemplary embodiment.

FIG. 9C is a top-down schematic view of springs usable with the joint of FIG. 9A, in accordance with one exemplary embodiment.

FIG. 10A is a side view of a portion of a robotic assembly, showing a joint and a spring and a linkage device, in accordance with one exemplary embodiment.

FIG. 10B is a side view of a portion of a robotic assembly, showing a joint and a spring and a linkage device, in accordance with one exemplary embodiment.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the singular forms “a,” and, “the” include plural referents unless the context clearly dictates otherwise. Thus, for example, reference to “a robotic arm” includes one or more of such robotic arms and reference to a “degree of freedom” (DOF) includes reference to one or more of such DOFs (degrees of freedom).

An initial overview of the inventive concepts is set forth below, and then specific examples are described in further detail in the Detailed Description. This initial summary is intended to aid readers in understanding the examples more quickly, but is not intended to identify key features or essential features of the examples, nor is it intended to limit the scope of the claimed subject matter.

The present disclosure sets forth a robotic assembly comprising a first joint comprising first and second support members rotatably coupled together; and a joint position restoration assembly coupled to at least one of the first or second support members, and comprising a first spring, the joint position restoration assembly being operable to apply a restoring torque to the first joint, wherein the joint position restoration assembly is configured to apply the restoring torque based on a current operating joint position within the restoring torque versus joint position profile relative to the first joint that corresponds to known mass properties of at least a portion of the robotic assembly acting on the first joint, such that, when the first joint is not undergoing powered actuation, the joint position restoration assembly operates to apply the restoring torque to position and to support the first joint in a stable support position.

In one example, the joint position restoration assembly can further comprise a mechanical linkage coupled between the first spring and at least one of the first or second support members, the mechanical linkage and the first spring operating together to facilitate application of the restoring torque to restore the first joint to the stable support position.

In one example, the first spring can comprise a linear spring and the mechanical linkage can further comprise a linkage device that operates with the linear spring to form, at least in part, the mechanical linkage.

In one example, one end of the first spring can be coupled to the first support member and the other end of the first spring can be coupled to the linkage device.

In one example, the mechanical linkage can comprise at least one of a simple crank, a four-bar linkage, a planetary transmission, or a roller screw.

In one example, the first spring and the mechanical linkage are collectively configured to provide a restoring torque versus joint position profile and the resulting restoring torque, relative to the first joint.

In one example, the first spring can comprise one of a pneumatic spring, a negator spring, a constant force spring, a linear spring, a rotary spring, a leaf spring, an elastomer spring, a composite flexural spring, a torsion bar, or a coil spring.

In one example, the first spring can be continuously engaged to apply the restoring torque independent of the rotational position of the first joint.

In one example, the robotic assembly can further comprise a second joint comprising the second support member and a third support member rotatably coupled to the second support member; and a second joint position restoration assembly coupled to at least one of the second or third support members, and comprising a second spring, the second joint position restoration assembly being operable to apply a restoring torque to the second joint, wherein the second joint position restoration assembly is configured to apply the restoring torque based on a restoring torque versus joint position profile relative to the second joint that corresponds to known mass properties of at least a portion of the robotic assembly acting on the second joint, such that, when the second joint is not undergoing powered actuation, the second joint position restoration assembly operates to apply the restoring torque to position and to support the second joint in a stable support position.

In one example, the robotic assembly can further comprise a third joint comprising the first support member and a fourth support member rotatably coupled to the first support member; and a third joint position restoration assembly coupled to at least one of the first or fourth support members, and comprising a third spring, the third joint position restoration assembly being operable to apply a restoring torque to the third joint, wherein the third joint position restoration assembly is configured to apply the restoring torque based on a restoring torque versus joint position profile relative to the third joint that corresponds to known mass properties of at least a portion of the robotic assembly acting on the third joint, such that, when the third joint is not undergoing powered actuation, the third joint position restoration assembly operates to apply the restoring torque to position and to support the third joint in a stable support position.

In one example, each of the first, second and third joint position restoration assemblies can be configured to correspond to known mass properties of the robotic assembly acting on the first, second and third joints, respectively, such that the first, second, and third joint position restoration assemblies are cooperatively operable to apply restoring torques to the first, second and third joints, respectively, to position and support the first, second, and third joints in respective stable support positions.

In one example, the first joint position restoration assembly can be coupled to the first and second support members, and the robotic assembly can further comprise a second joint comprising the second support member and a third support member rotatably coupled to the second support member; a third joint comprising the third support member and a fourth support member rotatably coupled to the third support member; and a second joint position restoration assembly coupled to the second and the fourth support members, and comprising a second spring, the second joint position restoration assembly being operable to apply a restoring torque to the second and third joints, wherein, when the first, second and third joints are not undergoing powered actuation, the first and second joint position restoration assemblies cooperatively operate to apply respective restoring torques to each of the first, second and third joints to position and to support the first, second and third joints in respective stable support positions.

In one example, the robotic assembly can comprise one of an exoskeleton, a humanoid robot, or a robotic limb. As such, the joints of the joint position restoration assemblies discussed herein can, but do not necessarily need to, facilitate movement in one or more degrees of freedom that correspond to one or more respective degrees of freedom of an element of a human body. Stated differently, the joints of the joint position restoration assemblies discussed herein may not facilitate movement of the robotic assembly in one or more degrees of freedom that correspond to a degree of freedom of an element of a human body.

In one example, the robotic assembly can further comprise a second joint comprising the second support member and a third support member rotatably coupled to the second support member, wherein the joint position restoration assembly is coupled to the first and third support members, the joint position restoration assembly being operable to apply a restoring torque to the first and second joints to position and to support the first and second joints in respective stable support positions.

In one example, the robotic assembly can comprise a lower body exoskeleton comprising a plurality of lower body support members, including the first and second support members, each rotatably coupled to at least one other support member; a plurality of joints defined by adjacently coupled lower body support members of the plurality of lower body support members; and a plurality of joint position restoration assemblies, each being operably coupled between at least two lower body support members defining at least one of the plurality of joints, such that the plurality of joint position restoration assemblies are cooperatively operable to position and support the plurality of joints in a stable support position.

In one example, the robotic assembly can comprise a full body exoskeleton, the lower body exoskeleton being operable with an upper body exoskeleton, the full body exoskeleton comprising a torso joint coupling the lower body exoskeleton to the upper body exoskeleton; and a joint position restoration assembly comprising a plurality of torso stabilizer springs coupled between the upper body exoskeleton and the lower body exoskeleton, and operable to position and support the upper body exoskeleton in a stable support position relative to the lower body exoskeleton.

In one example, the robotic assembly can comprise an exoskeleton, wherein the joint position restoration assembly can be operable to limit rotation of the first joint to prevent the first and second support members from collapsing when the exoskeleton is not operated by a user.

In one example, the joint position restoration assembly can further comprise a variable mounting position system that facilitates varying of a coupling location of the first spring to at least one of the first and second support members to vary (e.g., in one example optimize) the restoring torque provided by the joint position restoration assembly.

In one example, the robotic assembly can comprise an exoskeleton wearable by a user, wherein the first joint defines at least one degree of freedom corresponding to a degree of freedom of an element of a human body.

In one example, the exoskeleton can further comprises a plurality of joints, including the first joint, wherein at least some of the plurality of joints defines at least one degree of freedom corresponding to a degree of freedom of an element of a human body; a plurality of support members, including the first and second support members, wherein two or more of the plurality of support members are rotatably coupled together about respective joints of the plurality of joints; and a plurality of joint position restoration assemblies, including the joint position restoration assembly, each comprising a spring and a mechanical linkage operable with the spring, and each configured to provide a restoring torque versus joint position profile relative to one or more joints of the plurality of joints that corresponds to known mass properties of at least a portion of the robotic assembly acting on the one or more joints, respectively, wherein each of the plurality of joint position restoration assemblies is operable to apply a restoring torque to at least one of the one or more joints based on a current operating joint position within the restoring torque versus joint position profile to position and support the one or more joints in respective stable support positions.

In one example, the robotic assembly can further comprise a second joint defining a degree of freedom corresponding to a degree of freedom of an element of the human body, wherein the joint position restoration assembly is operable to apply collaborative restoring torques, including the restoring torque, to position and to support the first and second joints in respective stable support positions.

In one example, the robotic assembly can further comprise second and third joints operable with the first joint, and defining respective degrees of freedom corresponding to degrees of freedom of an element of the human body, wherein the joint position restoration assembly is operable to apply collaborative restoring torques, including the restoring torque, to position and to support the first, second and third joints to respective stable support positions.

The present disclosure also sets forth a robotic assembly comprising a first joint comprising first and second support members rotatably coupled together; a first joint position restoration assembly comprising a first spring, and operable to apply a restoring torque to the first joint; a second joint comprising the second support member and a third support member rotatably coupled to the second support member; a third joint comprising the first support member and a fourth support member rotatably coupled to the first support member; and a second joint position restoration assembly coupled to the second and the fourth support members, and comprising a second spring, the second joint position restoration assembly being operable to apply a restoring torque to the second and third joints, wherein, when the first, second and third joints are not undergoing powered actuation, the first and second joint position restoration assemblies cooperatively operate to apply respective restoring torques to each of the first, second and third joints to position and to support the first, second and third joints in respective stable support positions.

In one example, the first and second springs can be referred to as restorative springs operable to facilitate application of the respective restoring torques to restore the first, second and third joints to respective stable support positions, wherein the first and second springs are continuously engaged independent of the rotational positions of the first, second and third joints during operation of the exoskeleton.

In one example, each of the first and second joint position restoration assemblies can be configured to correspond to known mass properties of the robotic assembly acting on the first, second and third joints, respectively, such that the first and second joint position restoration assemblies are cooperatively operable to apply restoring torques to the first, second and third joints, respectively, to position and support the first, second, and third joints in respective stable support positions.

In one example, at least one of the first or second joint position restoration assemblies can further comprise a variable mounting position system that facilitates varying a coupling location of at least one of the first or second joint position restoration assemblies to vary the restorative and gravity compensation functions of the at least one of the first or second joint position restoration assemblies.

In one example, at least one of the first or second joint position restoration assemblies can further comprises a mechanical linkage.

The present disclosure further sets forth a method for configuring a robotic assembly, comprising configuring a first joint to comprise first and second support members rotatably coupled together; and configuring a joint position restoration assembly to be operable with the first joint, and to apply a restoring torque based on a restoring torque versus joint position profile relative to the first joint that corresponds to known mass properties of at least a portion of the robotic assembly acting on the first joint, such that when the first joint is not undergoing powered actuation, the joint position restoration assembly operates to apply the restoring torque to position and to support the first joint in a stable support position.

In one example, configuring the joint position restoration assembly to be operable with the first joint can comprise configuring the joint position restoration assembly to comprise a first spring and a mechanical linkage coupled between the first spring and at least one of the first or second support members, the mechanical linkage and the first spring operating together to facilitate application of the restoring torque to restore the first joint to the stable support position.

In one example, configuring the joint position restoration assembly to be operable with the first joint can comprise coupling a first end of the first spring to the first support member and coupling a second end of the first spring to the second support member.

In one example, configuring the joint position restoration assembly to be operable with the first joint can comprise coupling a first end of the first spring to the first support member and coupling a second end of the first spring to a linkage device of the mechanical linkage.

In one example, the method can further comprise configuring a second joint to comprise the second support member and a third support member rotatably coupled to the second support member; and configuring a second joint position restoration assembly to be operable with the second joint, and to apply a restoring torque based on a restoring torque versus joint position profile relative to the second joint that corresponds to known mass properties of at least a portion of the robotic assembly acting on the second joint, such that when the second joint is not undergoing powered actuation, the joint position restoration assembly operates to apply the restoring torque to position and to support the second joint in a stable support position.

In one example, the method can further comprise configuring a third joint to comprise the first support member and a fourth support member rotatably coupled to the first support member; and configuring a third joint position restoration assembly to be operable with the third joint, and to apply a restoring torque based on a restoring torque versus joint position profile relative to the third joint that corresponds to known mass properties of at least a portion of the robotic assembly acting on the third joint, such that when the third joint is not undergoing powered actuation, the joint position restoration assembly operates to apply the restoring torque to position and to support the third joint in a stable support position.

In one example, the method can further comprise configuring a second joint to comprise the second support member and a third support member rotatably coupled to the second support member; configuring a third joint to comprise the third support member and a fourth support member rotatably coupled to the third support member; and configuring a second joint position restoration assembly to be coupled to the second and the fourth support members, the second joint position restoration assembly being operable to apply a restoring torque to the second and third joints, wherein, when the first, second and third joints are not undergoing powered actuation, the first and second joint position restoration assemblies cooperatively operate to apply respective restoring torques to each of the first, second and third joints to position and to support the first, second and third joints in respective stable support positions.

In one example, the method can further comprise configuring a second joint to comprise the second support member and a third support member rotatably coupled to the second support member, and configuring the joint position restoration assembly to be operable with the first and second joints, and to apply respective restoring torques based on the restoring torque versus joint position profile relative to the first and second joints, such that the restoring torque versus joint position profile corresponds to known mass properties of at least a portion of the robotic assembly acting on the first and second joints, and such that when the first and second joints are not undergoing powered actuation, the joint position restoration assembly operates to apply respective restoring torques to position and to support the first and second joints in a stable support position.

In one example, the method can further comprise configuring the joint position restoration assembly to comprise a variable mounting position system that facilitates varying of a coupling location of the first spring to at least one of the first and second support members to vary (e.g. in one example optimize) the restoring torque provided by the joint position restoration assembly.

The present disclosure still further sets forth a robotic assembly, comprising a joint operable to undergo medial/lateral rotation between a stable support position and an actuated position; first and second support members rotatably coupled together about the joint; and a joint position restoration assembly comprising a spring, and coupled to at least one of the first or second support members, the joint position restoration assembly being operable to apply a restoring torque to the joint to return the joint from the actuated position to the stable support position.

In one example, the joint position restoration assembly can be configured to apply the restoring torque to the joint to return the joint in the stable support position upon powered actuation of the joint ceasing.

In one example, the spring can comprise a torsional spring.

In one example, the joint can define a degree of freedom that corresponds to a degree of freedom of medial/lateral rotation of a human leg.

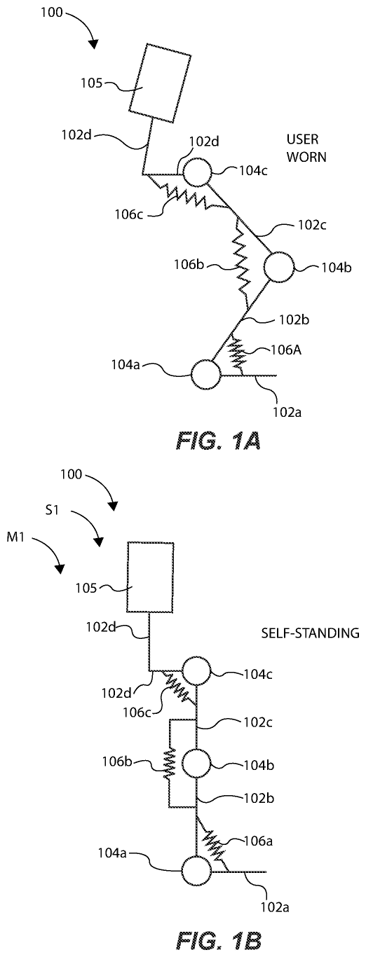

To further describe the present technology, examples are now provided with reference to the figures. FIGS. 1A and 1B schematically illustrate a robotic assembly, such as an exoskeleton 100, that is operable to achieve a stable support position S1, such as to support itself in an upright or standing position, such as when not in operation by a user (FIG. 1B), in accordance with an example of the present disclosure. The exoskeleton 100 can comprise one or more joint position restoration assemblies operable with one or more joints to position and/or support (i.e., maintain) the one or more joints in a stable support position.

As used herein, the term “joint” refers to the kinematic pairing of two or more links or structural components (e.g., 1) structural support members moveable (e.g., rotatable) relative to one another, 2) input and output members moveable relative to one another and as coupled to structural support members (e.g., those of an actuator, or actuator assembly (e.g., one or more actuators operable with a transmission, clutch, or other components, or a combination of these)), 3) any kind of base platform or a portion of an upper robotic assembly supported by the base platform) of a robotic system or a robotic assembly, or a combination of these, wherein a joint comprises both a structural connection (i.e., how the joint is formed, or rather the structural components making up the joint) and a functional connection (i.e., the degrees of freedom provided or facilitated by the joint). A joint can comprise various types, such as a lower pairing (e.g., revolute, prismatic, screw, cylindrical, universal, or planar), a higher pairing, or a combination of these. A joint can be part of a serial or parallel kinematic chain of links (and in some cases other joints). One or more joints can be arranged to be both in a parallel and a serial arrangement with respect to one or more other joints. A joints can be made to facilitate movement in one degree of freedom or multiple degrees of freedom depending upon its configuration.

As used herein, the term “stable support position” refers to a stabilized, self-supporting position of one or more joints and jointed structural members in a robot or robotic system or assembly, such as an exoskeleton (upper body, lower body/lower base or a combination of these), robotic arm, humanoid robot, teleoperated robot, robotic end effector, parallel mechanisms such as a Stewart platform (where the parallel mechanisms are referred to as joints or jointed structures as they collectively facilitate relative rotational movement between two object, systems, structures, etc.), and others, wherein all or a part of the robot or robotic system or assembly is able to support itself with the one more joints in these stable support positions under influence of an external force, such as a gravitational force (i.e., a gravity-induced torque acting on one or more joints), such as when not being operated or used under power (i.e., the joints are not undergoing powered actuation), as facilitated by the joint restoration assembly or assemblies associated with the joint(s). In other words, a stable support position refers to a position of one or more joints that are able to be maintained in that position without external sources, such as a gravitational forces, operating to rotate the joints (i.e., the joints are self-supporting in that position by overcoming gravitational forces). Example stable support positions can include a self-supporting standing, or self-standing, position (e.g., for a lower or full body exoskeleton), a self-supporting upright or default position (e.g., for an upper or full body exoskeleton, an upper robotic section supported about a base platform), a self-balancing position, or various other self-supporting positions that are not necessarily upright, such as those oriented on an incline relative to a vertical axis from a ground or other support surface. The stable support position can refer to the position of one or more joints of the robotic assembly, one or more structural members associated with one or more joints, or the entire robotic assembly (e.g., exoskeleton as a whole).

As used herein, a “joint position restoration assembly” refers to a system comprising one or more springs (i.e., restorative springs) and a mechanical linkage, these being operable with one or more “joints” of a robotic assembly (e.g., the exoskeleton 100) to provide a spring-based compensation of a gravity-induced load or torque in the form of a restoring torque to the respective joint(s) based on a restoring torque versus joint position profile that corresponds to the pre-determined or known weight or mass properties of at least a portion of the robotic assembly acting on or at the respective joints to position (i.e., restore) the respective joint(s) (and the components of the robotic assembly associated with or making up the respective joints) in/to a stable support position, and to support (i.e., maintain) the respective joint(s) in this position. Stated another way, the joint position restoration assembly refers to a system comprising one or more springs and a mechanical linkage that operates to stabilize (i.e., position and support) one or more degrees of freedom as provided or facilitated by one or more joints, such that the one or more joints and the associated structural components of the robotic assembly are in a stable support position. Indeed, a joint position restoration assembly can be operable to store and release energy and to continuously apply a restoring torque to one or more structural components to compensate for gravity-induced forces acting to move one or more structural components of the robotic assembly relative to one or more other structural components of the robotic assembly in the at least one degree of freedom. The joint position restoration assembly can further comprise linkage devices, mounting members or devices (e.g., mounting brackets) operable with or as part of the springs and/or mechanical linkages, and any other components necessary to operatively couple the joint position restoration assembly to the robotic assembly for its intended function as discussed herein. Some of these may facilitate adjustment of the one or more springs and/or the mechanical linkage, thus forming, at least in part, a variable mounting position system. A “restoring torque,” or simply a torque, refers to the rotating force generated by the joint position restoration assembly, which rotational force is applied to the joint to rotate the joint and the support member components of the joint to achieve the desired stable support position from a current operating position of the joint.

As used herein, a “restoring torque versus joint position profile” refers to a profile of the various joint position and/or robot position dependent rotating forces and/or torques that create a needed or desired restoring torque as generated by the joint position restoration assembly. A restoring torque versus joint position profile corresponds to the pre-determined or known weight or mass properties of at least a portion of the robotic assembly acting on or at a respective joint or joints. A joint or joints can have different restoring torques to be applied depending upon the position of the joint or joints.

As used herein, “mass properties” refers to any properties associated with the mass of all or part of the robotic assembly and/or robotic system that are associated with (i.e., acting on or at) a given joint or combination of joints, and that collectively operate to exert a gravity-induced torque at the joint. Mass properties can include, but are not limited to, the weight or mass of all or part of the robotic assembly or robotic system (including any objects coupled to or otherwise carried by or supported by the exoskeleton), any inertia, any forces or moments, any centers of mass, the angular position of the joint or joints, or other properties as will be apparent to those skilled in the art that act on, or that is/are experienced at, a particular joint or set of joints, and that contribute to the gravity-induced torque at the joint or set of joints.

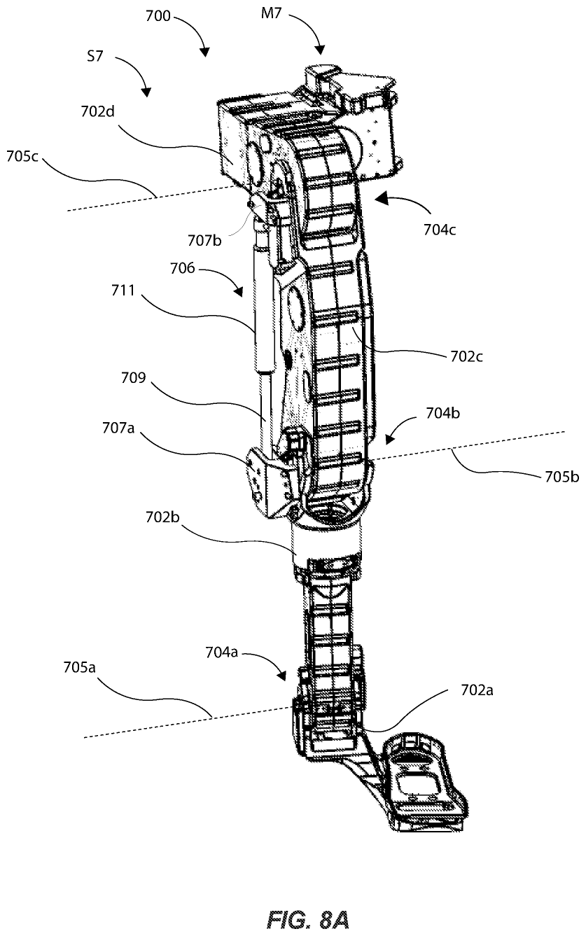

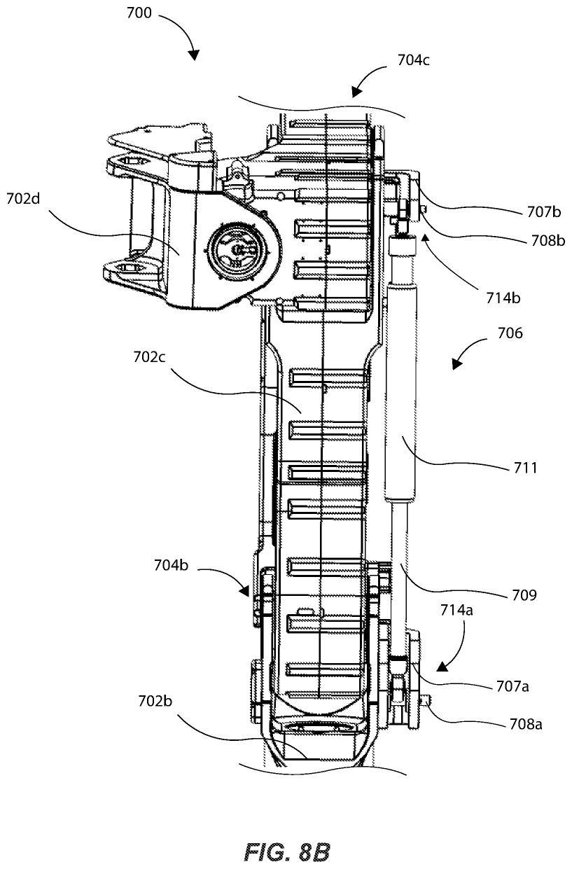

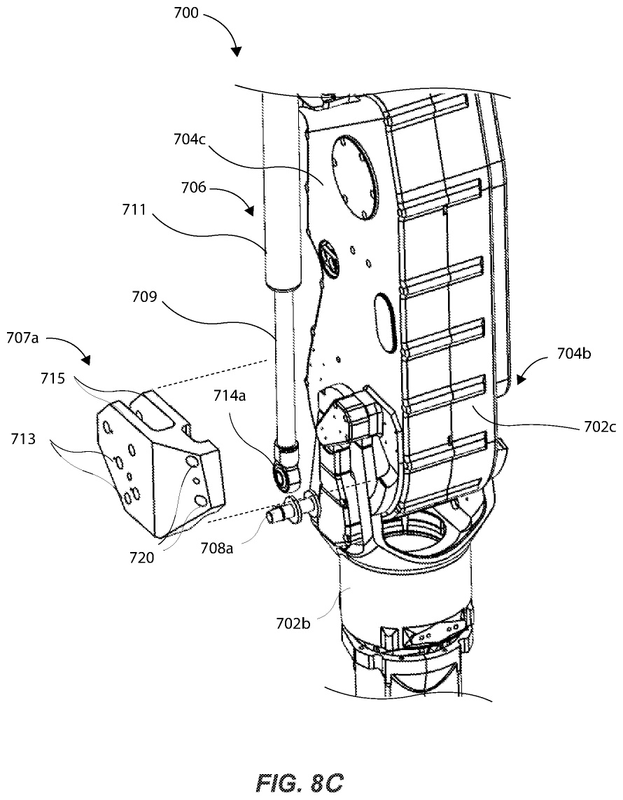

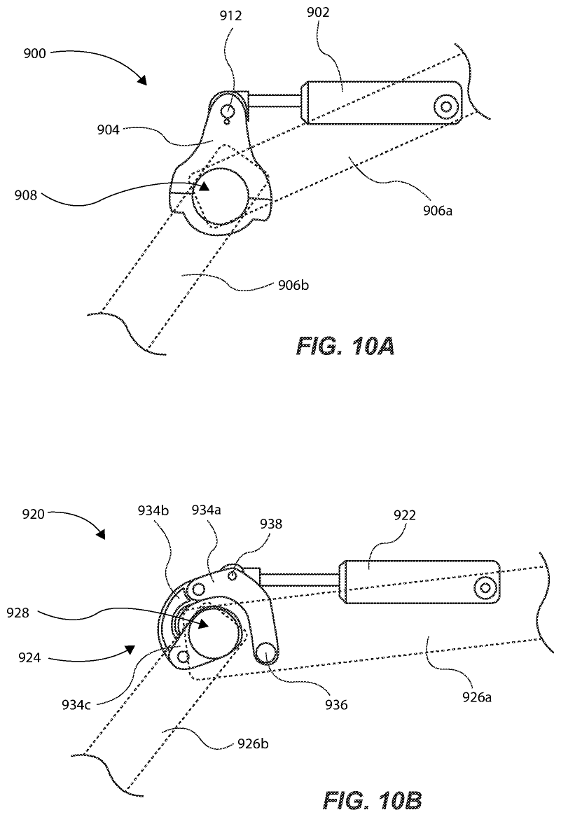

As used herein, the term “spring” refers to various types of energy storage and return elements. Example “springs” can include, but are not limited to, coil springs, elastomers, elastomer springs, polymer springs, leaf springs, helical springs, negator springs, constant force springs, pneumatic springs, rotary springs, composite flexural springs, torsion bars, or other suitable types. It is intended that the one or more “springs” be operably coupled to structural components on both sides of a joint by the mechanical linkage so as to effectuate rotation in one or more joints. The joint position restoration assembly can further comprise, and the one or more springs can be operably coupled to or a part of, one or more mechanical linkages, such as one configured to convert linear motion to rotary motion, rotary motion to rotary motion, rotary motion to linear motion, rotary motion to eccentric motion, and others. Various types of mechanical linkages are contemplated herein, such as, but not limited to, simple crank type of linkages, four-bar linkages, planetary transmissions, roller screws and others. In one example, the joint position restoration assembly can comprise a mechanical linkage formed simply from various structural support members of the robotic assembly (e.g., the exoskeleton 100) that are rotatable relative to one or more other support members about one or more joints, where the one or more springs are coupled directly to suitable support members of the robotic assembly. In some examples, the one or more springs can form a component of the mechanical linkage. It is noted that the support members can, in some examples, be adjacent one another, but this is not required. Indeed, in other examples, a single spring can be coupled to non-adjacent support members. Thus, a single joint position restoration assembly can provide a restoring torque to one joint or multiple joints, depending upon how it is configured. In another example, other linkage members and/or components not part of the support members of the robotic assembly can be part of the joint position restoration assembly, and can be used to facilitate the functionality of the joint position restoration assembly (e.g., see FIGS. 8A-8C, 9A-9C, and 10A-10B). The joint position restoration assembly, and particularly the mechanical linkage, can further comprise a mechanical linkage device (e.g., the mechanical linkage shown in FIGS. 10A-10B and described herein, or any others). As mentioned above, and as discussed in more detail below, the joint position restoration assembly can further comprise a variable mounting position system configured to facilitate fixed, or in other examples real-time (i.e., during operation of the robotic assembly by a user), adjustment and tuning of the one or more springs and/or the mechanical linkage of the joint position restoration assembly.

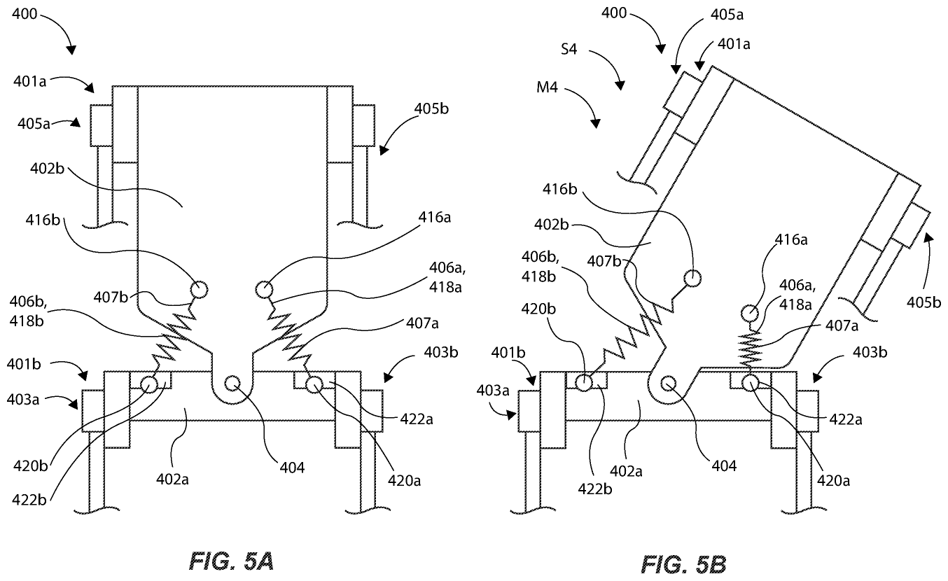

The exoskeleton 100 can comprise a plurality of support members (e.g., see support members 102a-d) rotatably coupled together. Respective support members can be rotatably coupled together to define and rotate about a plurality of joints (see joints 104a-d). In the case of the exoskeleton 100, for example, each joint can define a degree of freedom corresponding to a degree of freedom of an element of the human body, such as an ankle (e.g., joint 104a), a knee (e.g., joint 104b), a hip (e.g., joint 104c), and a torso (e.g., joint 104d) (see FIGS. 5A-7B for examples of a joint corresponding to a torso rotation). The joints identified above and shown in FIGS. 1A and 1B are simply illustrative of some of the joints that may exist in an exoskeleton (or other robotic system or assembly), and these are not meant or intended to be limiting in any way, nor are they intended to suggest that the exoskeleton 100 cannot have more or less than these. To the contrary, it is noted that in some cases, the exoskeleton can comprise more or less joints than those described above and shown in the drawings. In addition, the exoskeleton 100 can comprise one or more joints that represent a single joint of the human body. Stated differently, a joint of the exoskeleton 100 can be one of a plurality of joints that each define a degree of freedom, but that collectively correspond to a single element of the human body. Indeed, an exoskeleton can often comprise and utilize multiple joints, each comprising a degree of freedom, to mimic the multiple degrees of freedom achievable by a single joint of the human body. For example, it is contemplated that the exoskeleton 100 can comprise multiple joints that each define a different degree of freedom of a hip of the human body. Thus, the schematic illustration of joint 104c in FIGS. 1A and 1B may represent one, two, and/or three robotic joints associated with the various degrees of freedom of the human hip (i.e., (i) hip rotation with the axis of rotation generally along the long axis of the femur; (ii) hip flexion/extension that allows the leg to swing forward and back; and (iii) hip abduction/adduction). The same is true of an ankle of the human body (i.e., joint 104a may represent two or more robotic joints associated with the degrees of freedom of the human ankle). Accordingly, as will be appreciated by this disclosure and the examples below, one joint position restoration assembly with its spring may be used to support or be operable with one, two, or three robotic joints in stable support positions.

The exoskeleton 100 may comprise a backpack or torso structure 105 for supporting components that facilitate operation of the exoskeleton 100, such as a battery pack, computer, controllers, and other components, systems or devices. Note that a particular self-standing exoskeleton of the present disclosure can comprise a full body (combined upper and lower body exoskeleton) or just a lower body exoskeleton. Further note that each of the joints of the exoskeletons of the present disclosure can comprise a joint mechanism or module that includes an actuator of various types, for example, an electric actuator, a pneumatic actuator, a hydraulic actuator, or any others as will be apparent to those skilled in the art for facilitating actuation and rotation of the joint during use, as well as other possible components operable with the actuator, such as quasi-passive elastic actuators, potential energy storage devices, sensors, gear trains, transmissions, clutches, and others, as will be recognized by those skilled in the art.

Each support member 102a-d can be rotatably coupled to at least one other or one additional support member to form and define a respective joint. In other words, a joint can comprise adjacent support members from the various support members 102a-d rotatably coupled together to (at least partially) to form and define and rotate about respective joints. For instance, support members 102a and 102b can be rotatably coupled together about and can form and define a first joint 104a, which provides the exoskeleton 100 with a degree of freedom corresponding to a degree of freedom of an ankle joint of a human leg for flexion/extension of the foot about the first joint 104a (as noted above, this ankle location on the exoskeleton may include two or more joints, and one spring 106a may be operable with one or more robotic joints). Similarly, support members 102b and 102c can be rotatably coupled together about and can form and define a second joint 104b, which provides the exoskeleton 100 with a degree of freedom corresponding to a degree of freedom of a knee joint of the human leg for flexion/extension about the second joint 104b. Support members 102c and 102d can be rotatably coupled together about and can form and define a third joint 104c (or multiple joints at this hip location), which provides the exoskeleton 100 with a deuce of freedom corresponding to a degree of freedom of a hip joint of the human leg for flexion/extension about the third joint 104c. Although specific support members and respective joints of the exoskeleton 100 are set forth, this is not intended to be limiting in any way. Indeed, it is noted that the present disclosure anticipates and contemplates a variety of different types and configurations of robots and robotic systems and assemblies that can benefit from the technology described herein, and such technology is contemplated for use on these variety of different types and configurations. Indeed, although the examples provided below are examples of exoskeleton type robots or robotic systems, this is not intended to be limiting in any way. The present technology is equally applicable to other types of robots and/or robotic systems, such as humanoid robots, teleoperated robots, robotic arms, robotic end effectors, parallel mechanisms such as a Stewart platform, and other types as will be apparent to those skilled in the art.

It is noted herein that the first, second, and third joints 104a-c shown in the drawings and discussed herein, are not limited to defining one or more degrees of freedom of the robotic assembly corresponding to a one or more degrees of freedom of an element of a human body. Indeed, any combination of or all of such joints can operate within a robotic assembly to define one or more degrees of freedom of the robotic assembly, which do not correspond to any degree of freedom of an element of a human body. Therefore, the example of an exoskeleton and any joints of a joint position restoration assembly that do define one or more degrees of freedom that correspond to one or more degrees of freedom of an element of the human body is not meant to be limiting in any way. This is true of any of the joints discussed herein that are part of any of the example joint position restoration assemblies.

The exoskeleton 100 can include various components that, in various combinations have mass properties (e.g., mass properties M1) that act on or at each respective joint and that, because of gravity forces, cause a resulting gravity induced torque to be applied at each respective joint. The various mass properties acting on or at each joint can be determined prior to designing/tuning a particular joint position restoration assembly with its spring (and any associated linkage device, etc.), so as to ensure that any joint position restoration assembly including one or more springs and linkage configurations associated with a joint of the exoskeleton 100, for the purposes discussed herein, can be configured, and in some cases adjusted (i.e., tuned), to apply a spring-based compensation of a gravity-induced load or torque in the form of a restoring torque to the respective joints based on a current operating position of the joint within the restoring torque versus joint position profile, which profile corresponds to the pre-determined or known mass properties of at least a portion of the exoskeleton acting on the respective joints. Tuning of the joint position restoration assembly can be accomplished in one or more ways, such as tuning of the spring stiffness value of the spring(s), mounting the spring(s) and any associated linkage or linkage device in various locations relative to the joint, and others. As understood in the art, mass properties for one or more joints can be determined based on a number of different parameters and/or factors for a particular component or assembly of components, such as density, volume, mass center of gravity, inertia, rotational position, etc. For purposes of clarity, the mass properties M1 (and others like M2, M3, etc.) are merely shown for illustration purposes in the drawings, and therefore it should be appreciated that the particular “mass properties” associated with or acting on any particular joint(s) of a robotic assembly (for purposes of designing and configuring a joint position restoration assembly for particular joint(s) one or more possible restoring torque versus joint position profile(s) that correspond to the mass properties of at least a portion of the exoskeleton acting on the respective joints) can vary depending on the location and type and configuration of joint(s), and other variables such as support structure size, shape, material, mass, the rotational range of support structures, etc. Still other variables can contribute to the mass properties and gravity induced torque associated with a particular joint of exoskeleton 100, such as resistance from clutches, transmissions, etc. that may be incorporated in a particular robotic joint that would vary the restoring torque versus joint position profile required for a particular spring (and the associated mechanical linkage) to support a portion/section/assembly of the exoskeleton and the joint(s) in a stable support position. In one aspect of a simplified example, the mass properties M1 can be determined relative to at least one of the joints 104a-c, such that the corresponding joint position restoration assembly with its spring and any associated mechanical linkage can be configured to support or restore an associated one of the joints 104a-c in/to a stable support position that facilitates the exoskeleton 100 being supported in/to a self-standing configuration.

With respect to all of the examples discussed herein and shown in the drawings, it is to be understood that various factors can be taken into consideration when designing/tuning a particular joint position restoration assembly see FIGS. 10A and 10B as comprising a mechanical linkage having a linkage device) for positioning and supporting one or more robotic joints in a stable support position, such as the mass properties (e.g., of an exoskeleton, humanoid robot, etc.) associated with a particular joint, the spring type and material, the spring stiffness or K value, the spring equilibrium position (i.e., the zero force point), etc., and other parameters, such as the kinematics and kinetics of any incorporated linkage devices, transmissions, etc. that may operate with the spring of the joint position restoration device and the robotic joint to achieve the desired stable support position of the one or more joints, which stable support position can include a self-supporting position of the robotic assembly (e.g., an exoskeleton, humanoid robot, etc.) when not being operated under power. Mass properties relevant to a particular joint or combination of joints can also comprise a range depending upon the angular or rotational position of the joints. As such, each of the mass properties discussed herein and the resulting gravity-induced torques applied at each joint or combination of joints can comprise a range depending upon the angular or rotational position of joint or combination of joints.

As indicated above, the exoskeleton 100 can include a plurality of joint position restoration assemblies, each with one or more springs (e.g., see the respective joint position restoration assemblies with springs 106a-c), each one being operably coupled to respective adjacent support members about a joint (note that a spring can instead be coupled to one support structure and to a linkage device that is coupled to one or both support members; see e.g., FIGS. 10A and 10B and the discussion herein, which can be incorporated here and applicable to the exoskeleton 100). In the schematic example exoskeleton 100 shown, springs 106a-c can be operably coupled to respective adjacent support members 102a-d to apply a restoring torque to respective joints 104a-c to cause the joints 104a-c and the exoskeleton to move to an upright position, namely the stable support position S1, and/or to support (i.e., maintain) the exoskeleton 100 in the stable support position S1. This may be considered a restorative position of the exoskeleton 100 and the springs 106a-c as the springs operate to overcome the influence of external forces, such as gravity induced torque experienced at a particular joint due to the particular mass properties of at least a portion of the exoskeleton associated with the joints 104a-c. The springs 106a-c can be positioned, such that the restoring torque applied to respective joints 104a-c is generated by a rotational force applied to the joints 104a-c through the support members in support of the springs 106a-c to cause the joints 104a-c to rotate in the desired direction and to stabilize in a desired position. Depending upon the configuration of the exoskeleton 100, the location of the springs 106a-c relative to the rotational axis of each of the joints 104a-c and other characteristics of the springs 106a-c can be varied or different so as to provide a moment arm suitable for applying the correct restorative rotational forces for rotating the joints 104a-c in the correct direction, namely in the direction so as to achieve the stable support position S1.

Generally speaking, based on the center of gravity and the gravity-induced torque at each joint of the joints 104a-c at any given joint angle or position caused by the downward, gravitational forces exerted by the components at, near, and/or each joint of the joints 104a-c, a restoring torque can be calculated and implemented or applied for each joint of the joints 104a-c that reacts the loading (i.e., the gravity-induced torque) at each joint for a given position of the joint. In one example, this can be provided by the springs 106a-c. Each spring 106a-c can have a spring stiffness value (and other designed parameters) suitable to provide a spring-based compensation of gravity-induced load or torque present and acting at one or more joints of the joints 104a-c. Indeed, each joint position restoration assembly with its spring 106a-c can be selected and configured, and in some examples tuned, to provide a suitable restoring torque that reacts the gravity-induced loading present at a respective joint of the joints 104a-c that is based on the mass properties of a portion of the exoskeleton as associated with a particular joint. It is noted that gravity-induced loads can result from the mass properties associated with the joint, such as, but not limited to, the mass or weight of all or part of the exoskeleton, the location of such mass relative to a joint (the center of gravity of the mass the joint), as well as any other structures, objects, systems, devices present or existing on or otherwise associated with the exoskeleton. It is further noted that a gravity-induced load or torque can vary (i.e., be greater or less at different times) based on the position of the various support members and the rotational positions of the various joints of the exoskeleton 100 (i.e., the mass properties associated with each joint can change with the position of the joint, thus affecting the magnitude of the gravity-induced torque). As such, the selection, design, placement, and applied restoring torque (i.e., the tuning) of each of the joint position restoration assemblies with their springs 106a-c can take into account the different mass properties at, near, and/or above each different joint. Moreover, the restoring torque applied by each joint position restoration assembly designed to restore a given joint to a desired position (e.g., to achieve the stable support position S1) will need to be greater than the gravity-induced torque exerted as a result of the mass properties of the exoskeleton associated with each respective joint at a given angle or joint position in order to account for friction of the various components of the joints 104a-c (e.g., transmissions, clutches, etc.). It should also be pointed out that the attachment of the springs 106a-c is relevant, inasmuch as it has a direct connection then there will be more restoring torque than is needed in one region of the range of motion of each joint of the joints 104a-c to insure that there is a sufficient amount of restoring torque throughout all of the regions within the full range of motion. Alternatively, the actuator(s) (e.g., motor(s)) actuating the joints 104a-c, or even the operator if still donning the exoskeleton 100, could be employed to make up the difference.

In still another alternative, as mentioned above, the exoskeleton 100 can comprise a simple or complex linkage to provide closer to a desired restoring torque versus joint position profile across the full range of motion for one or more of the joints 104a-c. This could lead to higher design costs as well as increased weight, but these could be offset by being able to use a smaller spring since its output force would be used optimally across the full range of motion rather than making it strong enough to be suitable throughout the entire range of motion without an advantage of a linkage. Examples of a complex mechanical linkages having a linkage device are provided below regarding the discussion of FIGS. 10A and 10B, however, these are not intended to be limiting in any way, and it is contemplated that other mechanical linkages and linkage devices will be apparent to those skilled in the art.

As indicated above, each spring 106a-c can have a spring stiffness value and the joint position restoration assemblies with their respective springs 106a-c and any associated kinematic linkages can be configured, and in some cases tuned, to create a desired torque versus joint position profile suitable to provide a spring-based compensation of gravity-induced load or torque present and acting at a respective joint of the joints 104a-c, wherein the springs 106a-c and any linkage kinematics can each be can be configured, and in some cases tuned, to correspond to the mass properties M1 of the exoskeleton 100 associated with each respective joint, such that the springs 106a-c and any linkages are operable to counteract and overcome any degree of gravity-induced torques to restore and/or support (i.e., maintain) the exoskeleton 100 in the stable support position S1 (which stable support position S1 can comprise any desired position throughout a range of operating positions of one or more joints) via the restoring torques applied to the joints 104a-c by the springs 106a-c. Regarding the “tuning” of each joint position restoration assembly with respective springs 106a-c, if the mass properties M1 of the portion of the exoskeleton 100 associated with each respective joint are known, then each joint 104a-c may have a particular “gravity-induced torque” about or at the particular joint (at a particular rotational angle) that is experienced at each joint, for example, when the joint is not powered and when the exoskeleton 100 is not operated by a user (or supported by some other external force), and is under the influence of gravitational forces. The gravity-induced torque applicable at each joint can take into account any inherent resistance existing at the joint (e.g., due to friction, bearings, gear train, cogging torque of an EM motor, and other such factors). In essence, as used herein, “gravity-induced torque” can be described as the torque experienced at a particular powered-off joint subject to gravitational forces exerted due to the mass properties from one or more structural support members 102a-d, the torso structure 105, and/or other relevant components of the exoskeleton 100 that may contribute to the mass properties M1, which gravity-induced torque tends to cause the joint to rotate. For instance, in one non-limiting example, assume a maximum gravity-induced torque of joint 104c (hip joint) is 20 Nm/degrees, which is calculated based on the mass properties acting on or otherwise associated with the joint 104c, and which correspond to the particular angular displacement of the joint 104c at which it is desired for the spring 106c to position and to support (i.e., maintain) the joint 104c in that particular position. Based on this, the joint position restoration assemblies with its spring 106c can be can be configured, and in some cases tuned, so as to generate or meet a suitable restoring torque versus joint position profile (configured or tuned to generate a suitable restoring torque at this particular rotational position of the joint 104c) (e.g., tuning of the spring stiffness value, tuning of the configuration of the spring 106c, and tuning in regards to how the restoring spring 106c is operably coupled to the joint) sufficient to provide and apply a restoring force or torque (again at any given joint rotational position) that counteracts the 20 Nm/degrees gravity-induced torque of the joint 104c (but that does not apply too much torque to over rotate or actuate support member 102d) in such a way that the spring 106c is able to counteract or overcome the gravity-induced torque and achieve a restorative position suitable to position and to support (i.e., maintain) the joint 104c in the stable support position S1 of FIG. 1B (a restorative position of the joint 104c) via the restoring torque applied by the spring 106c. The joint position restoration assemblies and spring 106c can be configured, and in some cases tuned, to provide a restoring torque greater than this, but in any event, one that is sufficient to return the joint 104c to a restorative position in light of the mass properties M1. If the joint position restoration assemblies and spring 106c are configured or tuned such that the restoring torque is insufficient to place the joint 104c in a restorative position, such as to generate a restoring torque of only 15 Nm/degrees in this example, then the gravitational forces may cause the joint 104c to rotate or over rotate, wherein the support member 102d collapses or rotates too far toward the ground. On the other hand, if the spring 106c is “too stiff,” such as to provide a restoring torque of 40 Nm/degrees, then the joint 104c may be caused to rotate an undesirable amount, so that the support member 102d over rotates upwardly, which may make the exoskeleton unbalanced, and thereby prone to falling over/forward. In addition, an increased and unnecessary amount of work would need to be done by the active actuator to actuate the joint 104c during normal use and operation of the exoskeleton 100 to overcome the restoring torque produced by the restoring spring 106c. Therefore, the restoring torque value generated by the restoring spring 106c is intended to be selected so as to be as minimal as possible while still being able to achieve the restorative functions and positions described herein. This selective tuning can take into account the mass properties of the exoskeleton associated with the joint 104c, and desired angles of rotation of the joint 104c, as well as the final restorative position intended for the joint 104c, which final restorative position (the stable support position) can be varied in some examples. Alternatively, one or more mechanical stoppers can be incorporated into the joint 104c to provide a hard stop to prevent over rotation of the joint 104c in response to the restorative motion.

Those skilled in the art will recognize that the same principles discussed for joint 104c apply to the tuning or selecting of spring (and possible linkage device) characteristics for the joint position restoration assemblies and respective springs 106a and 106b to generate suitable respective restoring torques to rotate or actuate the respective joints 104a and 104b, and to support (i.e., maintain) them (and the support members defining these) in the stable support position S1 (FIG. 1B), and thereby restricting or prohibiting rotation of respective joints 104a and 104b once in the stable support position S1. In this manner, all three joint position restoration assemblies with respective springs 106a-c can be configured, and in some cases tuned, to cooperatively and collectively cause the joints 104a and 104b and the exoskeleton 100 to move to an upright or erect position, namely the stable support position S1, which can be referred to as a cooperative stable support position as it involves more than two support members and more than two joints, and to support (i.e., maintain) the exoskeleton 100 in this position when not operated by an operator (a non-operational state of the exoskeleton 100).

Note that the angular position of the respective support members relative to the respective joints 104a-c, when in the stable support position S1 can be any desired, and the torque required to restore any or all of the given joints 104a-c to achieve the stable support position S1 can be greater than or equal to the gravity-induced torque exerted by the mass properties acting on or otherwise associated with each joint of the joints 104a-c at a given angle. However, as noted above, the restoring torque for each of the springs 106a-c will likely be greater than the gravity-induced torque in order to overcome friction of the various components. As mentioned above, in one example, the respective support members relative to the respective joints 104a-c, when in the stable support position S1, can be positioned against limit stops defining a limit of travel of the joints 104a-c. Stated differently, the stable support position S1 can comprise the various support members or components of the joints 104a-c being positioned against physical hard stops that are part of the exoskeleton that limit rotation of the joints 104a-c beyond a certain angular position. In one aspect, this can be accomplished by a plurality of individual or single springs operably connected to the support members defining each respective joint of the joints 104a-c (i.e., a single spring 106a or a multi-spring arrangement connected to the support members 102a and 102b defining the joint 104a, a single spring 106b or multi-spring arrangement connected to the support members 102band 102c defining the joint 104b, and a single spring 106c or a multi-spring arrangement connected to the support members 102d and 102e defining the joint 104c), and acting to move each respective joint of the joints 104a-c. In another example, an arrangement of springs made up of one or more springs can be used to restore the joints to the desired stable support position S1, which can comprise a desired neutral position. The spring arrangement can be configured, such that it would not be possible to position the support members beyond or outside of the neutral position when the springs have reached their full restorative capacity.

The restorative capacity of each spring 106a-c can depend on the particular designed torque versus joint position profile of the various joints and the needed or desired restoring torques to be generated by the joint position restoration assemblies with respective restoring springs 106a-c. In this example, such a profile can be based on, for instance, the spring stiffness or spring constant, the configuration and the mounting locations of the restoring springs 106a-c, as well as the kinematics of the linkages they are operable with (which in this example is the various respective support members of the joints of the exoskeleton). The restoring torque versus joint position profile can take into account any limit stops associated with each joint (and its respective support members) that restrict or prohibit rotation of one or more joints once stabilized by the springs 106a-c in the stable support position S1.

The springs of FIGS. 1A and 1B, and the other springs exemplified herein, can be considered “restorative” springs, meaning that they can compensate or assist in compensating for gravity-induced torques or loads acting at the joints and can be utilized, along with a mechanical linkage, to apply the restoring torque, based on a restoring torque versus joint position profile, to position (i.e., restore) or maintain (or both) the joints of an exoskeleton and the support members associated with the joints in a stable support position (e.g., the exoskeleton is restored to, and maintained in, an upright or standing position) without active (e.g., braked) or powered assistance (e.g., no assistance from respective joint actuators is necessary, although such assistance from one or more actuators could be recruited), wherein the restorative forces (i.e., the restoring torques) applied by the springs operate to overcome external forces acting on the joints of the exoskeleton, such as gravity induced torques, having a tendency to collapse the joints (when unpowered or unclutched/braked). Springs and their configurations can operate to prevent the collapse of the joints of the exoskeleton under the influence of external forces. As indicated above, it is contemplated that the actuator of a robotic joint associated with a given degree of freedom can easily (i.e., using very little power) be recruited, if needed, to correct for any small discrepancy that might exist between actual position and/or torque and the desired position and/or torque of a robotic joint and its support members (such as in the stable support position). Indeed, using one or more sensors (e.g., force sensors, position sensors, or both) associated with a joint, the exoskeleton can be configured to sense and determine the position of the joints to determine if the restoring torque provided by the springs was sufficient to place the joint and the support members in the correct position. If not, then the actuator can be caused to further actuate the joint to achieve the correct position that has been pre-determined, such as a pre-determined stable support position.

It should be appreciated that the particular “known mass properties” corresponding to at least a portion of a particular robotic assembly (e.g., an exoskeleton) and that are associated with a respective joint will depend on the positions of the particular joint through its range of motion relative to the mass of the components associated with the particular joint. For instance, the relevant “mass properties” of the joint 104a of the exoskeleton for purposes of tuning the joint position restoration assembly with its spring 106a and mechanical linkage characteristics to create a restoring torque versus joint position profile that corresponds to the mass properties acting on or otherwise associated with the joint 104a can include the calculated mass of all of the components acting on the joint 104a, such as the mass of support member 102b-d, torso structure 105, and any other component supported by such support members 102b-d and torso structure 105, and their position relative to the joint 104a through the full rotational range of movements of the joint 104a in order to account for any gravity induced torques at each joint at different rotational positions of the joint 104a. The restoring torque versus joint position profile for a given joint can be designed to be based on the largest or maximum gravity-induced torque to potentially be experienced at the joint so that a suitable restoring torque can be generated throughout the full rotational range of the joint. However, this is not intended to be limiting in any way as the restoring torque versus joint position profile can be based on any gravity-induced torque to potentially be experienced at the joint, such as through a less than full rotational range of the joint, or in the event of recruitment of the joint actuator for partial active actuation by the actuator at the joint. It is noted that the weight of support member 102a may not be part of the “mass properties” associated with the joint 104a of the exoskeleton 100 because the ground may support the weight of the support member 102a.

Note that a particular robotic assembly, such as an exoskeleton, of the present disclosure can be a full body (upper and lower body exoskeleton) or just a lower body exoskeleton. As indicated above, each of the joints of the exoskeletons of the present disclosure can comprise a joint mechanism or module that includes one or more electric, pneumatic, or hydraulic actuators (or other types) for facilitating actuation and rotation of the joint during use, and other components operable with the actuator(s), such as sensors, gear trains, clutches, brakes, and other devices, systems or components.

Further note that the angular position of the respective support members relative to the respective joints 104a-c, when in the stable support position S1, will depend on the particular designed restoring torque versus joint position profile provided for the respective joints 104a-c (and may further depend on limit stops associated with a joint).

As will be appreciated from the examples discussed herein, the term “self-standing” exoskeleton (or humanoid robot) can refer to a particular type of stable support position. Self-standing can mean that a particular exoskeleton (e.g., only a lower body exoskeleton, or a combination upper and lower body exoskeleton) can operate to achieve a stable support position (e.g., stable support position S1) where select joints of the exoskeleton are caused to be in a stable support position, such that the exoskeleton is able to stand erect or upright on its own relative to ground. In this position, gravitational forces acting on one or more joints of the exoskeleton having a tendency to generate a gravity-induced torque at the joints are overcome and insufficient to cause gravity-induced torque or rotation of the one or more joints, thus positioning (i.e., restoring) the exoskeleton in/to the self-standing stable support position with the exoskeleton upright relative to ground, wherein this position can be achieved and maintained without the assistance of the powered actuators in the respective joints, meaning that the exoskeleton, not being operated under power, can comprise the joints not undergoing any powered actuation. In some cases, this may include an exoskeleton operable to stand on its own and under its own support without the assistance of an external support, such as from a user, an overhead hanger, a ground-based platform, or other device or structure that may operate to support some or all of the weight of the exoskeleton. Thus, in some cases, the term or phrase “self-standing” can mean that the exoskeleton (or humanoid robot) can balance and can stand or be generally upright on its own (in some cases this can also mean that the exoskeleton is also self-balancing). However, this does not exclude the scenario in which an external support is used. Indeed, an external support may be used to provide at least some support (e.g., balance) to the exoskeleton. In one example, an external support may be used to help balance the exoskeleton once in the stable support position. In another example, the back torso area of an exoskeleton may be leaned against a wall. In this example, a spring associated with an ankle joint may not be required or needed, such as if the exoskeleton is leaned against a wall or other structure when the user exits the exoskeleton. In this configuration, only a spring associated with a knee joint may be required in cases where the back side of the torso area of the exoskeleton can lean against a wall or other structure. Another spring associated with a hip joint may be incorporated to further support the exoskeleton in a self-standing position while a back side of the exoskeleton is leaned against a wall or other structure. In any event, the term “self-standing” is simply intended to mean that the exoskeleton (including the joints and the support members associated with the joints) can be caused to achieve any stable support position desired in which gravity induced torques acting on the one or more joints of the exoskeleton are overcome, at least to some extent, such that one or more joints are restored and maintained in a stable support position positioning the exoskeleton upright relative to ground, wherein this stable support position can be maintained without requiring the exoskeleton to be receiving power, or without requiring active actuation of any powered actuator(s), if present, of the joint.

The exoskeletons exemplified herein can have a gravity compensation component by virtue of utilizing one or more joint position restoration assemblies having respective springs associated with one or more joints. More specifically, assume that a user (wearing an exoskeleton) crouches down and compresses the springs (e.g., 106a and 106b). The one or more joint position restoration assemblies comprising the springs and associated mechanical linkage(s) coupled to or otherwise operable with the springs 106a and 106b can be designed and configured (and in some examples adjusted or tuned) to generate suitable restoring torque versus joint position profiles that correspond to the mass properties of at least a portion of the exoskeleton associated with the respective one or more joints, and that facilitate application of spring-based restoring torques in the respective one or more joints that nearly or exactly compensates for the gravity induced torque exerted on the joints during crouching. Thus, when the user stands up, the springs can release stored energy to apply a restoring torque to the respective joints so that energy is recovered of the system. In this manner, an actuator (e.g., hydraulic or electromagnetic motor) associated with a joint for actuation can be smaller in size and capacity, because less active torque from such actuator is needed to actuate the joint in the opposite direction (e.g., during standing up) because the springs have applied an augmented or supplemental torque to assist with active rotation of the respective joints. As a result, less energy is expended, and less heat is generated, as compared to the same or similarly configured system without such springs (because a larger motor and/or higher gear ratio would be necessary to actuate the joint in the absence of the spring).

In one specific example of the benefits of gravity compensation, assume that an exoskeleton weighs 40 kg, and a user crouches down to pick up a 40 kg payload. As the user crouches, the joint position restoration assemblies with their respective springs will store energy and will nearly or exactly compensate for the gravitational forces acting on the exoskeleton. Once the user picks up the 40 kg payload, the joint position restoration assemblies with their respective springs will release enough energy to assist to lift 40 kg of the exoskeleton, while electromagnetic actuators at the joints (e.g., hip and knee joints) are operated to lift the 40 kg payload. Thus, because of the joint position restoration assemblies and springs, the actuators are only needed to lift the 40 kg, and not the additional 40 kg of the weight of the exoskeleton. Accordingly, in this simplified example, the actuators can be half the capacity than would be required without incorporation of the springs.

Another benefit of incorporating joint position restoration assemblies with their respective springs with an exoskeleton is that the system is safer, because if power is accidentally terminated to the exoskeleton, then the joint position restoration assemblies and springs can help hold the exoskeleton in a standing or upright position, which can prevent injury to the user (if the exoskeleton is being worn) and others in proximity to the exoskeleton.

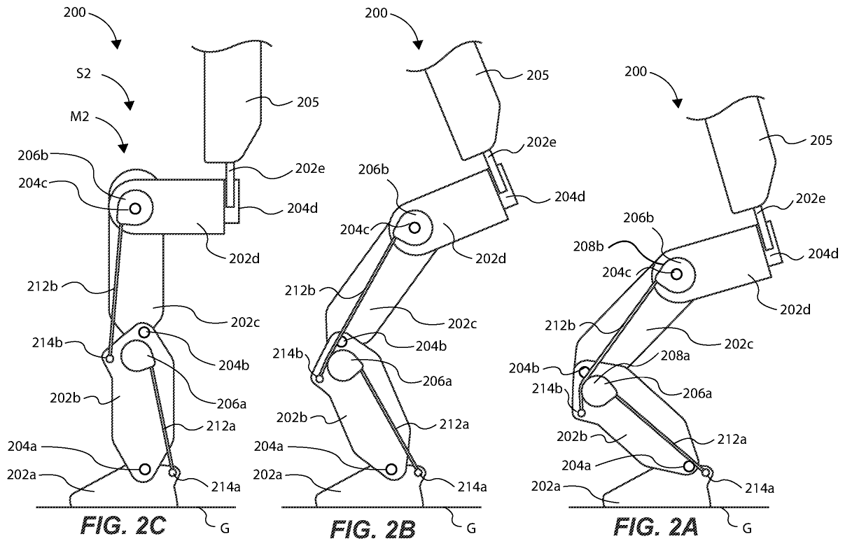

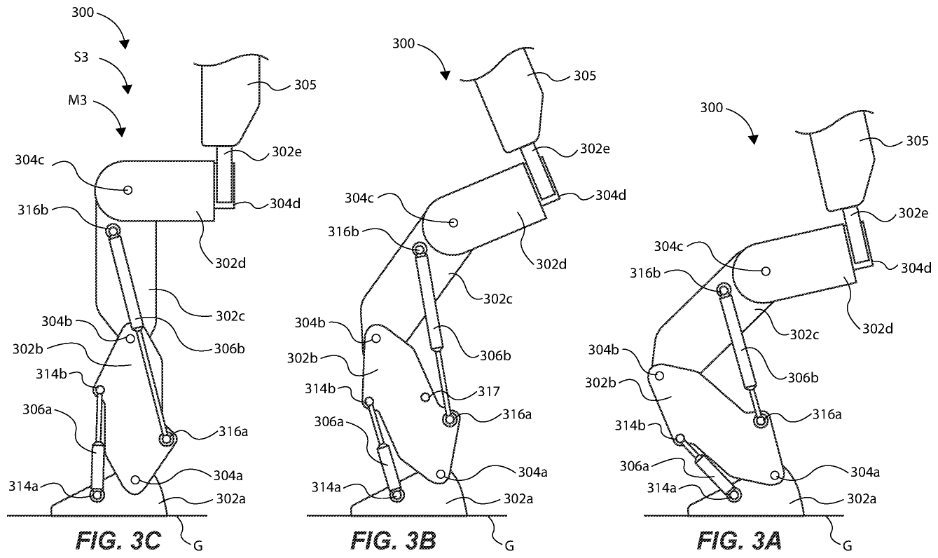

More specific examples of exoskeletons having one or more joint position restoration assemblies with one or more springs are set forth below. However, the discussion above pertaining to FIGS. 1A-1B is intended to be generic to each of the more specific exoskeleton examples discussed below, and to provide additional support to each of these. As such, the above discussion as it pertains to FIGS. 1A-1B is intended to be incorporated into the respective discussions of each of the example exoskeletons set forth below, where applicable, and where recognized by those skilled in the art. For purposes of clarity, FIGS. 1A, 2A, and 3A are meant to illustrate the respective self-standing exoskeletons as if they were being worn by (i.e., donned) or attached to a user or operator, while FIGS. 2B and 3B are meant to illustrate the respective self-standing exoskeletons in a transition position without a user present (i.e., the exoskeleton is not worn by or attached to the user), and while FIGS. 1B, 2C, and 3C are meant to illustrate the respective self-standing exoskeletons in their respective stable support positions (also with no user wearing the exoskeleton). Note that FIGS. 2A-7B illustrate relatively simplified exoskeletons for purposes of illustrating various spring and joint configurations, but it should be appreciated that these principles can be applied to a more complex exoskeleton than shown here, such as one that has three powered robotic joint mechanisms at or near the hip area to account for a fuller range of movement that may be necessary for three degrees of freedom associated with a human hip joint. And, as noted above, one or more joint position restoration assemblies with their respective springs (and possible linkage devices) may be implemented in a more complex manner to restore one or more robotic joints (e.g., powered hip joints) to a self-standing position.

It is further noted that, although robotic assemblies in the form of exoskeletons are specifically discussed and shown in the drawings, this is not intended to be limiting in any way with regards to the type of robotic assembly in which the present technology can be implemented. Indeed, it is contemplated that the present technology can be implemented in any type of robotic assembly, including, but not limited to, humanoid robots, robotic limbs, such as robotic arms, and any others as will be apparent to those skilled in the art.

FIGS. 2A-2C illustrate a robotic assembly, such as an exoskeleton 200, that is operable to achieve a stable support position S2, which in some examples, not being limited to this, can comprise a balanced, standing position (FIG. 2C) (thus being a self-standing exoskeleton), when not being actively actuated using power, in accordance with an example of the present disclosure. In the case of an exoskeleton, this can mean when the exoskeleton is not donned and in operation by a user, or when the joints are not being actively actuated even if donned by a user. Similarly as discussed above, the exoskeleton 200 can comprise a plurality of support members (e.g., see support members 202a-e) rotatably coupled together. Respective support members can be rotatably coupled together to rotate about and to (at least partially) form and define a plurality of joints (e.g., see joints 204a-d). In the case of the exoskeleton 200, each joint can define a degree of freedom corresponding to a degree of freedom of an element of the human body, such as an ankle (e.g., represented by joint 204a), a knee (e.g., joint 204b), a hip (e.g., represented by joint 204c) and a torso (e.g., joint 204d). In other words, the various joints (e.g., see joints 204a-d) can comprise support members rotatable relative to one another. The exoskeleton 200 may comprise a backpack or torso structure 205 for supporting components that facilitate operation of the exoskeleton 200, such as battery pack, computer, controllers, etc. Just as discussed above, the joints 204a-d can be part of a robotic assembly that is not an exoskeleton, and the joints 204a-c may not necessarily correspond to a degree of freedom of an element of the human body.

Each support member 202a-e can be rotatably coupled to at least one other or one additional support member to form and define a respective joint. In other words, a joint can comprise adjacent support members from the various support members 202a-e rotatably coupled together. For instance, support members 202a and 202b can be rotatably coupled together about and can form and define a first joint 204a, which provides the exoskeleton 200 with a degree of freedom corresponding to a degree of freedom of an ankle joint of a human leg for flexion/extension of the foot about the first joint 204a. Similarly, support members 202b and 202c can be rotatably coupled together about and can form and define a second joint 204b, which provides the exoskeleton 200 with a degree of freedom corresponding to a degree of freedom of a knee joint of the human leg for flexion/extension about the second joint 204b. And, support members 202c and 202d can be rotatably coupled together about and can form and define a third joint 204c, which provides the exoskeleton 200 with a degree of freedom corresponding to a degree of freedom of a hip joint of the human leg for flexion/extension about the third joint 204c. Finally, support members 202d and 202e can be rotatably coupled together about and can form and define a fourth joint 204d, which provides the exoskeleton 200 with a degree of freedom corresponding to a degree of freedom of a torso joint (e.g., side-to-side movement about the fourth joint 204d (see e.g., FIG. 5A)).

The exoskeleton 200 can include mass properties M2 acting on or otherwise associated with each respective joint, which can include the mass properties of some or all of the components of the exoskeleton 200. The exoskeleton 200 can comprise one or more joint position restoration assemblies, each comprising one or more springs (e.g., see springs 206a and 206b), each one being operably coupled to one or more support members about one or more joints, wherein some cases the springs are operable with a linkage device and linkage mechanism that are part of the joint position restoration assembly, and in some cases the springs are coupled directly to the support members, which then function as part of the joint position restoration assembly. In the example exoskeleton 200 shown, first and second joint position restoration assemblies comprising respective springs 206a and 206b can be operably coupled to respective support members 202a-d, as shown, where the first and second joint position restoration assemblies cooperatively operate to apply respective restoring torques to each of the joints 204a-c to position and to support (i.e., maintain) the exoskeleton 200 in the stable support position S2, which may be considered a restorative position of the exoskeleton 200 and the joints 204a-c. In this example, two joint position restoration assemblies are operable to position and support three joints in the stable support position S2 by applying restorative torques to each of the joints 204a-c. Each of the first and second joint position restoration assemblies, with their respective spring 206a and 206b (including their configuration, mounting location, equilibrium position, etc., as well as any linkages associated with these), can be configured, and in some cases tuned, to provide a restoring torque versus joint position profile that corresponds to the mass properties M2 of the exoskeleton 200 acting on or otherwise associated with each respective joint, such that the joint position restoration assemblies with their respective springs 206a and 206b are operable to collaboratively or collectively position and support (i.e., maintain) the exoskeleton 200 in the stable support position S2 via the restoring torques applied to the joints 204a-c by the springs 206a and 206b. Regarding the “tuning” of each joint position restoration assembly, if the mass properties M2 of the portion of the exoskeleton 200 acting on or otherwise associated with each respective joint are known, then each joint 204a-c may have a particular gravity-induced torque about or at the joint (at a particular rotational angle) that is experienced at each joint when the joint is not powered and when the exoskeleton 200 is not operated by a user (or supported by some other external force), and is under the influence of gravitational forces and parasitic torque. That is, the joint torque includes gravity-induced torque of each joint along with inherent resistance of the joint (e.g., due to friction from bearings and gear train, togging torque of an EM motor, and other such factors). The gravity-induced torque is the torque experienced at the particular joint due to gravitational forces exerted on the mass of one or more structural support members 202a-c, the torso structure 205, and other relevant components of the exoskeleton 200 that may contribute to the mass properties M2. For instance, in one non-limiting example, assume a maximum gravity-induced torque of joint 204c (hip joint) is 20 Nm/degrees, which is calculated based on the mass properties associated with the joint 204b (knee joint). Based on this, the joint position restoration assembly having the spring 206b can be configured or in some examples tuned, so as to generate a suitable restoring torque versus joint position profile (to facilitate generation and application of a suitable restoring torque at the particular rotational positions of the joints 204b and 204c) sufficient to apply a restoring force or torque that counteracts the 20 Nm/degrees gravity-induced torque at the joints 204b and 204c, so that the spring 206b can position (i.e., restore) to a restorative position and/or maintain in such a position both the joints 204b and 204c. The same principle applies to the tuning or selecting of joint position restoration assembly having the spring 206a (with the design of possible linkages) to position and to support (i.e., maintain) the joint 204a via a restoring torque applied by the spring 206a to restrict or prohibit rotation of the joint 204a once in the stable support position S2. In this manner, both springs 206a and 206b can cooperate to support the exoskeleton 200 in the stable support position S2. As indicated, and as applicable to the examples discussed herein, the joint position restoration assemblies are intended to operate when the robotic assembly is not being used under power to actuate the joints. This can mean not actively actuating any of the joints with a user donning the robotic assembly in the case of an exoskeleton, or when there is no user. Or, this can mean when the joints are not actively being actuated in the case of a humanoid, robotic arm, or other type of robotic assembly not an exoskeleton.

In this particular example, though not intending to be limiting in any way, the springs 206a and 206b can each comprise a (near) constant force spring in the form of a negator spring. However, other spring types can be utilized and are contemplated, such as the one or more of the different spring types set forth above. As such, the negator type springs shown are not intended to be limiting in any way. Indeed, the springs 206a and 206b can comprise pneumatic springs, coil springs, for example, or any other as discussed herein. Each of the negator springs can comprise a support housing (e.g., see respective support housings 208a and 208b) and an internal rotational spring operably coupled to a flexible cable or tether (e.g., see flexible cables 212a and 212b, respectively) that is wound inside the support housing 208b. In this manner, the internal rotational spring is configured to cause or exert a constant or continuous pulling force on the flexible tether 212b, such that a force is required to pull the flexible tether 212b from within the support housing 208b.

As shown, support housing 208a can be attached to or otherwise supported by support member 202b by suitable means (e.g., fastened), and support housing 208b can be attached to or otherwise supported by support member 202d by suitable means. The flexible tether 212a can be attached to a respective tether attachment mount 214a on support member 202a designed to receive, interface with and couple the flexible tether 212a, so that a constant or continuous restoring torque can be applied to the ankle joint 204a between the support members 202a and 202b. Likewise, the flexible tether 212b can be attached to a respective tether attachment mount 214b on support member 202b designed to receive, interface with and couple the flexible tether 212b, so that a constant or continuous restoring torque can be applied to the knee and hip joints 204b and 204c between the support members 202b and 202d. The support housing 208b can act as a pivot structural member to provide a pivot point to the flexible tether 212b to, so that the springs 206b can properly apply the restoring torque to both knee and hip joints 204b and 204c to maintain their respective positions. Thus, the spring 206b, being attached to support member 202d, is configured to apply restoring torques to the hip joint 204c and also to the knee joint 204b. This is because the tether attachment mount 214b is attached to the support member 202b adjacent the support housing 208a of the spring 206a, such that the support housing 208a is situated between the tether attachment mount 214b and the support housing 208b of the spring 206b, and also because support member 202c is linked between support member 202d (supporting the support housing 208b) and between support member 202b (supporting the tether attachment mount 214b). In one example, a particular attachment mount can comprise an eccentric surface that can provide a simple means to shape a restoring torque versus joint position profile as a function of joint position. Those skilled in the art will recognize that attachment points for the springs and the tether mounts can vary, or be different on different exoskeletons, humanoid robots, or other robotic assemblies, and as such, those illustrated in the figures and discussed above are not intended to be limiting in any way.

FIGS. 3A-3C illustrate a robotic assembly, such as an exoskeleton 300, that is operable to achieve a stable support position S3, which in some examples, not being limited to this, can comprise a balanced, standing position (FIG. 3C) (thus being a self-standing exoskeleton), when the joints are not being actively actuated, in accordance with an example of the present disclosure. In the case of an exoskeleton, this can mean when the exoskeleton is not donned and in operation by a user, or when the joints are not being actively actuated even if donned by a user. Similarly as discussed above, the exoskeleton 300 can comprise a plurality of support members (e.g., see support members 302a-e) rotatably coupled together. Respective adjacent support members can be rotatably coupled together to rotate about and to at least partially form and define a plurality of joints (e.g., see joints 304a-d). In the case of an exoskeleton, each joint can define a degree of freedom corresponding to a degree of freedom of an element of the human body, such as an ankle (e.g., joint 304a), a knee (e.g., joint 304b), a hip (e.g., joint 304c) and a torso (e.g., joint 304d). In other words, the various joints (e.g., see joints 304a-d) can comprise support members rotatable relative to one another. Just as discussed above, the joints 304a-d can be part of a robotic assembly that is not an exoskeleton, and the joints 304a-c may not necessarily correspond to a degree of freedom of an element of the human body. The exoskeleton 300 can have a backpack or torso structure 305 for supporting components that facilitate operation of the exoskeleton.

Each support member 302a-e can be rotatably coupled to at least one other or on additional support member to form and define a respective joint. In other words, a joint can comprise adjacent support members from the various support members 302a-e rotatably coupled together. For instance, support members 302a and 302b can be rotatably coupled together about and can form and define a first joint 304a, which provides the exoskeleton 300 with a degree of freedom corresponding to a degree of freedom of an ankle joint of a human leg for flexion/extension of the foot about the first joint 304a. Similarly, support members 302b and 302c can be rotatably coupled together about and can form and define a second joint 304b, which provides the exoskeleton 300 with a degree of freedom corresponding to a degree of freedom of a knee joint of the human leg for flexion/extension about the second joint 304b. And, support members 302c and 302d can be rotatably coupled together about and can form and define a third joint 304c, which provides the exoskeleton 300 with a degree of freedom corresponding to a degree of freedom of a hip joint of the human leg for flexion/extension about the third joint 304c. Finally, support members 302d and 302e can be rotatably coupled together about and can form and define a fourth joint 304d, which provides the exoskeleton 300 with a degree of freedom corresponding to a degree of freedom of a torso joint or movement of the human body, such as for side-to-side movement about the fourth joint 304d (see e.g., FIG. 5A).

The exoskeleton 300 can include mass properties M3 associated with each respective joint, which can include the mass properties of some or all of the components of the exoskeleton 300. As an overview, the exoskeleton 300 can comprise one or more joint position restoration assemblies, each comprising one or more springs (e.g., see springs 306a and 306b), each one being operably coupled to one or more support members about one or more joints, wherein some cases the springs are operable with a linkage device and linkage mechanism that are part of the joint position restoration assembly, and in some cases the springs are coupled directly to the support members, which then function as part of the joint position restoration assembly. In the example exoskeleton 300 shown, first and second joint position restoration assemblies comprising respective springs 306a-b can be operably coupled to respective support members 302a-c, as shown, where the first and second joint position restoration assemblies cooperatively operate to apply respective restoring torques to the joints 304a-b to position and to support (i.e., maintain) the exoskeleton 300 in the stable support position S3, which may be considered a restorative position of the exoskeleton 300 and the joints 304a-c. Each joint position restoration assembly with its respective springs 306a and 306b and any linkages can be configured, and in some examples tuned, so as to provide or facilitate a suitable restoring torque versus joint position profile that corresponds to the mass properties M3 of the exoskeleton 300 associated with each respective joint, such that the first and second joint position restoration assemblies are operable to counteract and overcome any gravity-induced torques to position (i.e., restore) and to support (i.e., maintain) the exoskeleton 300 to/in the stable support position S3 via the restoring torques applied to the joints 304a and 304b by the first and second joint position restoration assemblies with their respective springs 306a and 306b as based on the restoring torque versus joint position profile. Regarding the configuration or tuning of each joint position restoration assembly, if the mass properties M3 of the portion of the exoskeleton 300 associated with each respective joint are known, then each joint 304a-c may have a particular known gravity-induced torque about or at the joint (at a particular rotational angle) that is experienced at each joint when the joint is not powered and when the exoskeleton 300 is not operated by a user or operator, and is under the influence of gravitational forces and parasitic torque. That is, the joint torque includes gravity-induced torque of each joint along with inherent resistance of the joint (e.g., due to friction from bearings and gear train, cogging torque of an EM motor, and other such factors). The gravity-induced torque is the torque experienced at the particular joint due to gravitational forces exerted on the mass of one or more structural support members 302a-c, the torso structure 305, and other relevant components of the exoskeleton 300 that may contribute to the mass properties M3. For instance, in one non-limiting example, assume a maximum gravity-induced torque of joint 304b (hip joint) is 20 Nm/degrees, which is calculated based on the mass properties associated with the joint 304b. Based on this, the joint position restoration assembly having the spring 306b can be configured, or in some examples tuned, so as to generate a suitable restoring torque versus joint position profile sufficient to facilitate application of a restoring force or torque that counteracts the 20 Nm/degrees gravity-induced torque at the joint 304b, so that the spring 306b can position (i.e., restore) the joint 304b to a restorative position and maintain the joint 304b in such position. The same principle applies to the tuning or selecting of the joint position restoration assembly having the spring 306a (and possible linkages) to position (i.e., restore) and to support (i.e., maintain) the joint 304a in its stable support position via restoring torques applied by the spring 306a to restrict or prohibit rotation of the joint 304a once supported in the stable support position S3. In this manner, both springs 306a and 306b can cooperate together to position (i.e., restore) and to support (i.e., maintain) the exoskeleton 300 in the stable support position S3. As indicated, and as applicable to all of the robotic assembly examples discussed herein, the joint position restoration assemblies are intended to operate when the robotic assembly is not being used under power to actuate the joints. This can mean not actively actuating any of the joints with a user donning the robotic assembly in the case of an exoskeleton, or when there is no user. Or, this can mean when the joints are not actively being actuated in the case of a humanoid, robotic arm, or other type of robotic assembly not an exoskeleton.

One or more of the restorative springs 306a and 306b can comprise any of the spring types discussed herein, or that would be apparent to one skilled in the art. In the example shown, one or more of the springs 306a and 306b can comprise a linear spring in the form of a pneumatic spring, which includes a piston translatable through a support housing that contains a gas chamber, such as in the traditional configuration of a pneumatic compression spring. In another example, one or more of the springs 306a and 306b can comprise a linear spring in the form of a substantially balanced pressure pneumatic cylinder and piston, such as a commercially available nitrogen cylinder spring device, which may provide the advantage of maintaining a substantially constant spring force during the stroke of the nitrogen cylinder spring device. Other spring types can be utilized and are contemplated, such as the one or more of the different spring types set forth above. As such, the linear type springs shown are not intended to be limiting in any way. The spring 306a can have one end 314a pivotally coupled to the first support member 302a, and the other end 314b pivotally coupled to the second support member 302b (in a suitable manner of pivotally mounting an end of a pneumatic spring to a support member). Alternatively, one end of the spring (306a,b) can be coupled to a support member, and the other end can be coupled to a linkage device of a mechanical linkage that converts linear movement of the spring to rotational movement to rotate the joint (see e.g., FIGS. 10A and 10B, and the discussion below, which can be incorporated here). In this way shown in FIGS. 3B and 3C, the joint position restoration assembly having the spring 306a can apply a restoring torque to the joint 304a to rotate or actuate the joint 304a to the stable support position S3. For example, once the operator steps out of the exoskeleton 300, and the exoskeleton 300 is powered down so that its joint actuators are inactive, the spring 306a can release stored energy and expand to the position of FIG. 3B, and then to the position of FIG. 3A, thereby applying a restoring torque to position the joint 304a in the stable support position S3, and then to restrict or limit the rotation of joint 304a to prevent the support member 302b from collapsing under gravitational forces.

Similarly, the spring 306b can have one end 316a pivotally coupled to the second support member 302b, and the other end 316b pivotally coupled to the third support member 302c. Thus, the spring 306b can apply a restoring torque to the joint 304b to rotate or actuate the joint 304b to the stable support position S3. Again, once the operator steps out of the exoskeleton 300 and it is powered down, the spring 306b releases stored energy and expands to move the joint 304b to the position of FIG. 3B, and then to the position of FIG. 3A, thereby positioning and maintaining the joint 304a in the stable support position S3, and therefore applying a restoring torque to restrict or limit the rotation of joint 304b to prevent the support member 302c from collapsing under gravitational forces. Alternatively, the end 316b of the spring 306b can be pivotally coupled to the fourth support member 302d to apply a restoring torque to each of the second and third joints 304b and 304c (knee and hip joints).

In one example, and as part of the tuning function to provide a suitable restoring torque versus joint position profile, the mounting points of each spring 306a or 306b can be varied by way of a variable mounting position system, which can be part of the joint position restoration assembly, to vary (e.g., optimize) the restorative and gravity compensation aspects of the springs for a particular self standing exoskeleton, and even to accommodate different users having varying heights. For instance, the end 316a of the spring 306b can be mounted at a different position on the second support member 302b, such as at mounting point 317 (see FIG. 3B). Accordingly, the variable mounting position system can comprise a plurality of different discrete mounting points (e.g., 317, showing one option) located on the support member 302b for such purposes, which mounting points can comprise apertures formed through a mounting plate (see e.g., FIGS. 8A-8C) or other component of the second support member 302b. In another example, rather than separate discrete mounting points, the variable mounting position system can comprise a slot formed as part of the support member 302b (or formed on a support bracket (e.g., see FIGS. 8A-8C)), and then the end 316a of the spring 306b can be slidably coupled to the support member 302b via the slot, wherein the spring 306b can be slid to a desired position, and then locked into the desired position with a suitable locking device, such as via a fastener. In some cases, the variable mounting position system can vary the mounting position of the spring 306b in real-time, thus varying the restorative and gravity compensation aspects of the spring in real-time. In one example, the variable mounting position system can comprise an actuator (e.g., a motor, a pneumatic actuator, a fluid actuator, and others) operably coupled to the spring 306b, wherein the motor can be selectively actuated to vary the position of the spring 306b as described herein. Varying the mounting position of the spring can also function to vary the stable support position desired. For example, the stable support position S1 may be varied to include other positions in addition to the one illustrated in the figures. This same variable mounting positioning can be incorporated and implemented with any of the springs 306a or 306b (or any others). Moreover, although not described in detail in each of the example robotic assemblies set forth herein, it is noted that each of the different example robotic systems and assemblies described herein (FIGS. 1A-10B) can comprise a similar variable mounting position system as described here. In any case, the particular mounting position(s) of the spring(s) of the present disclosure can be varied or modified to suit a particular purpose and/or size of user.

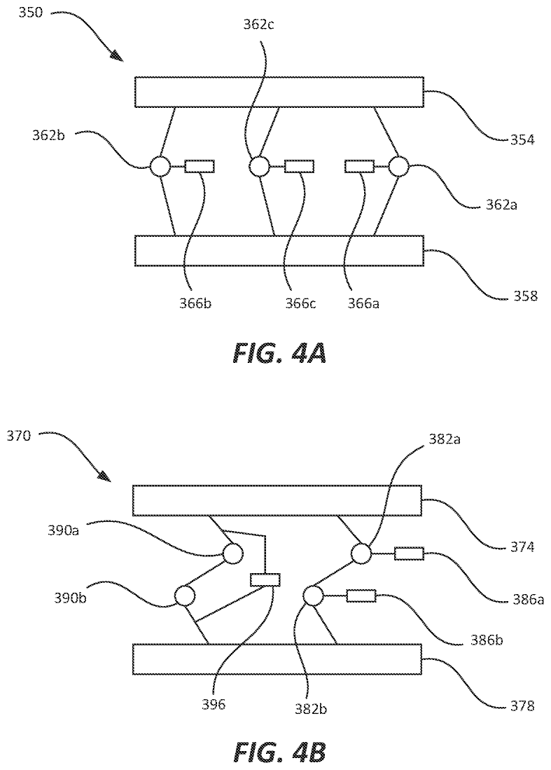

With reference to FIGS. 4A and 4B, the present disclosure further sets forth various robotic systems comprising an upper robotic assembly supported about a base platform, where the upper robotic assembly and the base platform are rotatable relative to one another via one or more joints in one or more degrees of freedom. FIGS. 4A-4B illustrate that the robotic systems disclosed herein can comprise any kinematic arrangement of joints, such as a serial kinematic chain of joints, a parallel kinematic chain of joints, or a combination of these. Specifically, FIG. 4A illustrates a schematic diagram representative of a robotic system 350 having an upper robotic assembly 354 rotatably coupled to a base platform 358 via one or more joints in parallel (e.g., six “joints” forming a Stewart platform, or other arrangements using between two and six joints in parallel), with the robotic system 350 further comprising one or more joint position restoration assemblies, each associated and operable with at least one of the one or more joints (e.g., see joint position restoration assemblies 366a, 366b, and 366c associated and operable with joints 362a, 362b, and 362c, respectively, used to represent two or more joints in parallel with one another) to place the one or more joints and/or the upper robotic assembly 354 in a stable support position relative to the base platform 358. The joint position restoration assemblies can be coupled to any suitable structural members in the robotic system. For example, one or more joint position restoration assemblies can be coupled to one or more of the various links or structural components of the robotic system associated with one or more joints. In another example, although not shown, one or more joint position restoration assemblies can be coupled to or between the upper robotic assembly and the base platform (such as providing one option for stabilizing a Stewart platform type of arrangement incorporated into the robotic system that facilitates relative movement between the upper robotic assembly and the base platform).

The upper robotic assembly 354 can comprise any type of robotic assembly, such as a robotic exoskeleton, a humanoid robot, a robotic limb, or any others as will be apparent to those skilled in the art. The base platform 358 can comprise any type of structural support member or system configured to support the upper robotic assembly 354. In one example, the base platform 358 can comprise a lower robotic assembly, such as a lower body exoskeleton (e.g., see FIGS. 1A-3C, 5A-7B, and 8A-8C), or any other type of robotic assembly.

In another example, the base platform 358 can comprise a mobile platform, capable of powered locomotion, or capable of being supported on a mobile vehicle. The mobile platform can comprise any type of vehicle or other mobile base. The mobile platform can comprise and support a power source, various drive system(s) to provide and facilitate locomotion and steering of the mobile platform, a pump, a generator, a fuel supply, a controller, user interface devices, communications systems (e.g., those facilitating remote or teleoperation of the upper robotic assembly 354 and the mobile platform), or any other device, object or system facilitating operation of both the mobile platform and the upper robotic assembly 354. In some cases, the mobile platform can further be configured to support one or more users. Thus, the upper robotic assembly 354 as supported by the mobile platform in accordance with the present disclosure can be a mobile, self-contained system capable of also supporting a user to operate the system. Indeed, the mobile platform can be configured to comprise or support all of the necessary elements, components, systems and/or subsystems to make up a fully or self-contained system that can be operated by the user and moved from location to location as desired.

In another example, the base platform 358 can comprise a boom or other moveable structure configured to position the upper robotic assembly 354 in a variety of positions within a zone of operation (i.e., the area in which the boom is able to locate or position the upper robotic assembly 354 via movement of the boom). The boom can be configured to move within a two or three-dimensional zone of operation. Moreover, the boom can be configured to move in up to six degrees of freedom, thus facilitating the positioning of the upper robotic assembly in a number of positions and orientations within a two or three dimensional zone of operation.

In still another example, the base platform 358 can comprise a fixed, stationary type of base platform for supporting the upper robotic assembly 354 in a fixed location. Those skilled in the art will recognize still other types of base platforms that could be used to support the upper robotic assembly 354. As such, those discussed herein are not intended to be limited in any way.

FIG. 4B illustrates a schematic diagram representative of a robotic system 370 having an upper robotic assembly 374 rotatably coupled to a base platform 378 via one or more joints, wherein the one or more joints and/or the upper robotic assembly 374 can be positioned in a stable support position via one or more joint position restoration assemblies, each operable with at least one of the two or more joints. The upper robotic assembly 374 and the base platform 378 can be the same or similar to those discussed above with respect to FIG. 4A. FIG. 4B illustrates that the one or more joints can be parallel to one another, in series with one another, or any combination of these in support of the upper robotic assembly 374 relative to the base platform 378. The robotic system 370 can further comprise a plurality of joint position restoration assemblies, each operable with one or more of the joints. In one example, the robotic system 370 can comprise a plurality of joint position restoration assemblies, at least some of these being associated and operable with at least one respective joint of the plurality of joints in a series relationship with one another (e.g., see first and second joint position restoration assemblies 386a and 386b associated and operable with first and second series joints 382a and 382b, respectively). In another example, the robotic system 370 can comprise one or more joint position restoration assemblies associated and operable with a plurality of joints in series (e.g., see joint position restoration assembly 396 associated and operable with first and second joints 390a and 390b in series). Indeed, a single joint position restoration assembly can be associated with one joint or a plurality of joints to apply respective restoring torques to the associated joints, similar in concept to the lower body joint position restoration assembly(ies) of the exoskeletons 100, 200 and 300, as discussed above. The joints 390a and 390b (with their joint position restoration assembly(ies)) are in series with respect to one another, and can further be in a parallel arrangement with the joints 382a and 382b (with their joint position restoration assembly(ies)), which joints 382a and 382b are also in series with respect to one another. The joint position restoration assemblies can be coupled to any suitable structural members in the robotic system. For example, one or more joint position restoration assemblies can be coupled to one or more of the various links or structural components of the robotic system associated with one or more joints. In other words, the one or more joint position restoration assemblies can be coupled to the structural members moveable relative to one another via a joint or multiple joints. As shown in FIG. 4B, the joint position restoration assembly 396 can be coupled to a first structural member (that is coupled to the upper robotic assembly 374 and a second structural member movable relative to the first structural member via a first joint 390a), as well as to a third structural member that is coupled to the base platform 378 and the second structural member, where the second and third structural members are moveable relative to one another via the second joint 390b. Similarly, as also shown in FIG. 4B, the joint position restoration assembly 386a can be coupled to a first structural member and a second structural member movable relative to the first structural member via the first joint 382a, and the joint position restoration assembly 386b can be coupled to the second structural member and a third structural member moveable relative to the second structural member via the second joint 382b. Alternatively, the one or more joint position restoration assemblies of the robotic system 370 can be coupled at one end to a structural member of a joint and to one of the upper robotic assembly 374 or the base platform 378 at another end. In another example, one or more joint position restoration assemblies of the robotic system 370 can be coupled directly to or between each of the upper robotic assembly 374 and the base platform 378 (such as providing one option for stabilizing a Stewart platform type of arrangement incorporated into the robotic system 370 that facilitates relative movement between the upper robotic assembly 374 and the base platform 378). Indeed, the robotic system 370 can comprise a plurality of joints, some of which are in a series relationship with one another, and some of which are in a parallel relationship with one another, or a combination of these arrangements, as illustrated in FIG. 4B. The parallel and series joints can be arranged in a variety of different configurations, and it is noted that the specific arrangements shown in FIGS. 4A and 4B are not intended to be limiting in any way. Moreover, the robotic assembly 370 can comprise a plurality of joint position restoration assemblies operable with one or more of the joints as taught herein. It is noted that the joint position restoration assemblies shown in FIGS. 4A and 4B can comprise any type of coupling, springs, and linkages as discussed herein, or that would be apparent to those skilled in the art.

Examples of such robotic systems comprising an upper robotic assembly rotatably connected to a base platform are discussed below with respect to FIGS. 5A-7B. It is noted herein that the robotic systems of FIGS. 4A-7B can comprise any type of upper robotic assembly, such as, but not limited to, a robotic exoskeleton, a humanoid robot, a teleoperated robot, a robotic limb (e.g., a robotic arm), or any other type of robotic assembly that can be rotatably coupled to a base platform, wherein these are rotatable relative to one another via one or more joints that facilitate rotation of the upper robotic assembly and the base platform relative to one another in one or more degrees of freedom. Further note that, in a more advanced robotic assembly, it may not be apparent or distinct where a particular upper robotic assembly and a base platform begins or ends, because of the number of joints and support members that may be needed to effectuate rotation in one, two, or three degrees of freedom, for instance. Thus, the drawings and descriptions herein are not intended to be limiting in this regard, and it is possible that a particular joint facilitating movement (rotation) between an upper robotic assembly and a base platform in at least one degree of freedom may be part of and associated with support members of either the upper robotic assembly or the base platform, or both, and/or any additional support members supported between these. Moreover, a robotic system having an upper robotic assembly and a base platform rotatable relative to one another can be configured with a plurality of joints and respective support members, such that the upper robotic assembly and the base platform are moveable relative to one another via the plurality of joints in a plurality of degrees of freedom. All or some of these plurality of joints can be operable with a joint position restoration assembly. Each joint position restoration assembly can comprise one or more springs and associated mechanical linkages, as taught herein, wherein each of the joint position restoration assemblies operates to apply a restoring torque to one or more associated or respective joints as taught herein. Stated differently, each of the joint position restoration assemblies discussed herein can be operable to store and release energy and to continuously apply a restoring torque to the one or more joints, and thus effectively to the upper robotic assembly relative to the base platform, to compensate for gravity-induced forces acting to move the upper robotic assembly relative to the base support in one or more degrees of freedom. The joint position restoration assemblies, upon being coupled between the upper robotic assembly and the base platform to any arrangement of structural members (including portions of the upper robotic assembly and the base platforms themselves, if necessary or desired), can each be configured to apply a restoring torque to one or more joints and the degrees of freedom provided by such joints based on a restoring torque versus joint position profile relative to the one or more degrees of freedom that corresponds to mass properties of at least a portion of the robotic system associated with the one or more degrees of freedom, such that the joint position restoration assemblies each operate to apply a restoring torque to position and to support the upper robotic assembly in a stable support position relative to the base platform.

FIGS. 5A and 5B illustrate a robotic system comprising an upper robotic assembly and a base platform rotatably coupled together via one or more joints configured to facilitate movement of the upper robotic assembly relative to the lower robotic assembly in at least one degree of freedom. In the example shown, the robotic system is configured as a robotic exoskeleton 400, with the upper robotic assembly comprising an upper type of exoskeleton body section, and the base platform comprising a lower type of exoskeleton body section, wherein the upper and lower body exoskeleton sections together define the full body exoskeleton 400. The exoskeleton 400 is operable to support itself in a standing or upright position (including the upper body exoskeleton section being supported in an upright or centered, default position relative to the lower body exoskeleton section), which in this example comprises a stable support position S4 (FIG. 5A) that can be achieved by the below discussed joint position restoration assembly, such as when the exoskeleton 400 is powered down and/or not operated by a user, in accordance with an example of the present disclosure.

More specifically regarding FIGS. 5A and 5B, the robotic system in the form of the robotic exoskeleton 400 can comprise an upper body exoskeleton section 401a (or simply an upper body exoskeleton 401a) rotatably coupled to a lower body exoskeleton section 401b (or simply a lower body exoskeleton 401b) via at least one joint (e.g., see joint 404), such that these are rotatable relative to one another in one or more degrees of freedom. The joint 404 can comprise an actuatable joint. The lower body exoskeleton 401b can comprise a first support member 402a (i.e., lower body support member), which can support left and right leg members or sections 403a and 403b (such as leg members or sections of any of the lower body exoskeletons shown herein, or others). The upper body exoskeleton 401a can comprise a second support member 402b (e.g., upper body support member) rotatably coupled to the first support member 402a by way of, and that defines, a joint 404 configured to move in at least one degree of freedom, so as to facilitate movement in at least one degree of freedom of the upper body exoskeleton 401a relative to the lower body exoskeleton 401b. Indeed, as those skilled in the art will recognize, the joint 404 can be configured to move in two or more degrees of freedom, for example two or more rotational degrees of freedom, such that the upper and lower body exoskeletons 401a, 401b are able to move relative to one another in two or more degrees of freedom.

In another example, the upper body exoskeleton 401a can comprise additional support members (e.g., additional upper body support members) rotatably coupled to additional support members of the lower body exoskeleton 401b, and that are part of and that define a second joint (not shown) that defines and that is configured to move within at least one degree of freedom of movement, and that facilitates movement of the upper body exoskeleton 401a relative to the lower body exoskeleton 401b in at least one degree of freedom (and is some examples two or more degrees of freedom). The first and second joints can be in a parallel or series arrangement. The first joint 404 and the second joint, with their respective support members, can operate together to support the upper and lower body exoskeletons 401a, 401b, and to facilitate their rotation, relative to one another. This example illustrates that the upper and lower body exoskeletons 401a and 401b can be rotatably coupled to one another via a plurality of joints. Although not discussed in detail, each of these joints can be operable with a respective joint position restoration assembly (or two or more joints can be operable with a single joint position restoration assembly) where the one or more joint position restoration assemblies can comprise similar elements as, and that can function similar to, the joint position restoration assembly discussed below with respect to joint 404 to apply respective restoring torques to the respective first and second joints. In one aspect, the first joint can define a degree of freedom that corresponds to a first degree of freedom of a human torso, and the second joint can define a degree of freedom that corresponds to a second degree of freedom of a human torso. In still another example, the upper body exoskeleton 401a can comprise additional support members (e.g., additional upper body support members) rotatably coupled to additional support members of the lower body exoskeleton 401b, and that are part of and that define a third joint (not shown) that defines at least one degree of freedom of movement of the upper body exoskeleton 401a relative to the lower body exoskeleton 401b. The first joint 404, the second joint, and the third joint, with their respective support members, can operate together to support the upper and lower body exoskeletons 401a, 401b, and to facilitate their rotation, relative to one another. This example illustrates that the upper and lower body exoskeletons 401a and 401b can be rotatably coupled to one another via a plurality of joints, each able to be configured to move in one or more degrees of freedom. Although not discussed in detail, each of the first, second and third joints can be operable with a respective joint position restoration assembly (or a single joint position restoration assembly can be operable with the second and third joints), where each joint position restoration assembly can comprise similar elements as, and that can function similar to, the joint position restoration assembly discussed below with respect to joint 404 to apply independent and respective restoring torques to the respective first, second and third joints. In one aspect, the first joint can define a degree of freedom that corresponds to a first degree of freedom of a human torso, the second joint can define a degree of freedom that corresponds to a second degree of freedom of a human torso, and the third joint can define a degree of freedom that corresponds to a third degree of freedom of a human torso.

In the case of the exoskeleton 400, the joint 404 can define a degree of freedom corresponding to at least one degree of freedom of an element of a human body, such as lateral or side-to-side torso motion, and/or forward-to-back torso motion, and/or rotational torso motion in any combination, or a combination of all of these. In this regard, note that joint 404 in the drawings is a schematic representation of a joint that may rotate in one, two, or three degrees of freedom. Further note that more than one joint (i.e., a plurality of joints) may be incorporated to facilitate rotation of the upper body exoskeleton 401a relative to the lower body exoskeleton 401b, such as a joint situated above joint 404 and that is wholly supported by support member(s) of the upper body exoskeleton 401a for twisting rotation, for instance. As indicated above, in any robotic system, such as humanoid or others, the joint 404 does not necessarily need to define one or more degrees of freedom corresponding to one or more degrees of freedom of an element of the human body. Indeed, the joint 404 can define and facilitate movement of the upper and lower body exoskeletons 401a and 401b of the exoskeleton 400 relative to one another, whether these correspond to one or more degrees of freedom of an element of the human body or not. This is true for all of the joints in this or any of the other examples discussed herein, whether the joints are part of a lower or upper body portion of a robotic system. In one example however, the upper body exoskeleton 401a can comprise a torso section, wherein the second support member 402b of the upper body section is included within the torso section, such that the first joint defines a degree of freedom corresponding to a degree of freedom of rotation of the torso section of the human body.

The second support member 402b can support a backpack or other structural support for supporting components that facilitate operation of the exoskeleton 400, such as batteries, computers, controllers, and other devices, systems or components. The second support member 402b can support left and right exoskeleton arms 405a and 405b. Note that the second support member 402b is shown as a single structural support member, but this is not intended to be limiting in any way as the second support member 402b can be comprised of a number of different structural members or components coupled together to form a torso area or portion of an upper body exoskeleton.

The exoskeleton 400 can include mass properties M4 acting on or otherwise associated with each respective joint, which in this case can include the mass properties of the components of the robotic system associated with the joint 404, including the left and right exoskeleton arms 405a and 405b, that generate a gravity induced torque within the joint 404 that may tend to rotate or otherwise move the upper body section out of or away from a centered, default position. The exoskeleton 400 can comprise one or more joint position restoration assemblies, each having one or more springs and a mechanical linkage (with or without a linkage device). In this example, the exoskeleton 400 comprises a joint position restoration assembly comprising first and second springs 406a and 406b (e.g., torso stabilizer springs), operably coupled to respective adjacent support members 402a and 402b and operable with the joint 404 to apply respective restoring torques to the joint 404 (e.g., which can be an active, actuatable robotic joint or a passive robotic joint). The joint position restoration assembly with its springs 406a and 406b can further comprise at least one of a mechanical linkage (which in some examples can be adjustable), which linkage can further comprise a linkage device that operates with the spring to form, at least in part, the mechanical linkage. A spring stiffness of the springs 406a and 406b (which in some examples can be adjustable), an equilibrium position (i.e., zero force and/or torque position) (which in some cases can be adjustable), and any other characteristic or property of the joint position restoration assembly can be configured, or in some examples tuned, to generate a suitable restoring torque versus joint position profile that corresponds to the mass properties M4 acting on or at the joint 404, and that facilitates application of a spring-based restoring torque that compensates for the gravity-induced load or torque at the joint 404, such that the joint position restoration assembly with its springs 406a and 406b are each operable to position (i.e., restore) and to support (i.e., maintain) the upper body exoskeleton 401a in any desired position (i.e., the stable default position S4), such as a generally upright or vertical position (the centered, default position shown in FIG. 5A), via the restoring torques applied to the joint 404 by the joint position restoration assembly with its springs 406a and 406b. For instance, if the mass properties M4 associated with the joint 404 would exert a gravity-induced torque at the joint 404 of 20 Nm/degrees in the position shown, then the joint position restoration assembly and spring 406a (and similarly spring 406b) can be configured, or in some examples tuned, so as to generate a suitable restoring torque versus joint position profile sufficient to facilitate application of a restoring force or torque that counteracts and overcomes the 20 Nm/degrees of gravity-induced torque at the joint 404, so that the joint position restoration assembly with its springs 406a and 406b can both position and support the joint 404 in the stable default position S4, which therefore restricts or prohibits rotation of the joint 404, such as in the direction shown in FIG. 5A. Note that each spring 406a and 406b can be configured so as to not over compress or bottom out during movement of the joint 404. It is noted that the springs 406a and 406b can be configured to facilitate application of respective restoring torques to the joint 404, which joint 404 can be configured to move in two or three rotational degrees of freedom. In other words, the first spring 406a can be configured to facilitate application of a restoring torque to the joint 404 in a first degree of freedom, and the second spring 406b can be configured to facilitate application of a restoring torque to the joint 404 in a second degree of freedom.

The springs 406a and 406b can each comprise an elastic element configured to store and release energy and can comprise any of the types of springs discussed herein and identified above (or others as will be apparent to those skilled in the art), such as elastomers, an elastomer spring, a polymer, a leaf spring(s), a helical spring, a negator spring, a substantially constant force spring, a pneumatic spring, a rotary spring, a composite flexural spring, a torsion bar, or others. The springs 406a and 406b can include mounting portions 416a and 416b, respectively, which are mounted or attached to the second support member 402b, or a linkage device can be coupled between an end of a spring and a support member (see e.g., FIGS. 10A and 10B and the discussion herein, which can be incorporated here) as part of the joint position restoration assembly.

The joint position restoration assembly, with its associated restoring torque versus joint position profile, can be configured, and in some examples tuned, to allow the upper exoskeleton 401a to rotate to the left and right about joint 404, such as when the exoskeleton 400 is worn and operated by a user, so that the springs 406a and 406b can compress to a certain degree and not substantially interfere with the intended use of the exoskeleton (and also act as a gravity compensation component, such as discussed above). Thus, during use of the exoskeleton 400, the second support member 402b may be rotated to the left or right about the joint 404, such as during a lifting task with one arm. If the second support member 402b rotates to the right, as shown in FIG. 5A, the first spring 406a will compress to a certain degree and will store energy, thereby compensating for gravity acting on the upper body 401a. Meanwhile, the second spring 406b, depending upon its type and mounting arrangement, can be configured to extend to store energy, thereby cooperatively working with the first spring 406a to apply the restorative torque and compensate for gravity acting on the upper body 401a. Then, as the user rotates to the left (such as during picking up an item with a right arm), the first spring 406a (and possibly the second spring 406a) will release energy, which assists with actuation and rotation of the joint 404. In this way, a smaller actuator (e.g., a smaller electromagnetic motor) may be used to rotate the joint 404 than would otherwise be needed without incorporation of the springs 406a and 406b (in the example where the joint 404 is powered). Moreover, if the user steps out of the exoskeleton 400 and the upper body 401a happens to be leaning to the left or right (of the center, default position of FIG. 5A), the springs 406a and 406b can cooperate to position (i.e., restore) and to support (i.e., maintain) the upper body 401a in a generally upright, default support position, as shown in FIG. 5B. Thus, the springs 406a and 406b can each apply a restoring torque to the joint 404 to restore and maintain the position of the joint 404 and the second support member 402b in the stable support or default position S4. Said another way, the first and second springs 406a and 406b are situated on either side of the joint 404, such that the compression of the springs 406a and 406b occurs on either side of the joint 404. In this configuration, the first and second springs 406a and 406b cooperate to position (i.e., restore) and to support (i.e., maintain) the second support member 406b in an equilibrium position (FIG. 5B) to restrict bi-directional rotation of the joint 404 (i.e., the springs act to restore the joint position to its equilibrium position when the actuator does not apply a torque to the joint).

In another example, and as indicated above, the joint 404 can be configured to move in two or more degrees of freedom, with the first spring 406a configured to facilitate application of a first restoring torque to the joint by the joint position restoration assembly in a first degree of freedom of movement, and the second spring 406b configured to facilitate application of a respective second restoring torque to the joint by the joint position restoration assembly in a second degree of freedom of movement. In this example, the first and second springs 406a and 406b operative in a cooperative manner to support the exoskeleton 400 in the stable support position, which can be the default position shown in FIG. 5A.

Note that the lower body exoskeleton 401b may be configured like any of the examples discussed herein, or others. In this manner, the lower and upper body exoskeletons, defining a full body exoskeleton, can be a full body “self-standing” exoskeleton having a plurality of joint position restoration assemblies with respective springs and mechanical linkages (for lower body joints, and for the torso joint(s)) that cooperate together to position (i.e., restore) and to support (i.e., maintain) the exoskeleton in a stable support position (lower body), and the generally upright, default centered position (upper body) (these being stable support positions as discussed herein). Thus, when a user desires to re-use or again don the full body self-standing exoskeleton, the user can merely “step into” the exoskeleton 400 with comfort and ease because the exoskeleton 400 is already in a standing position or an upright position. Thus, an external system, such as a hoist or hanger, is not necessarily needed or required to support the exoskeleton 400 in such a position. This may be useful in applications where such external systems to support or hoist the exoskeleton are not readily available.

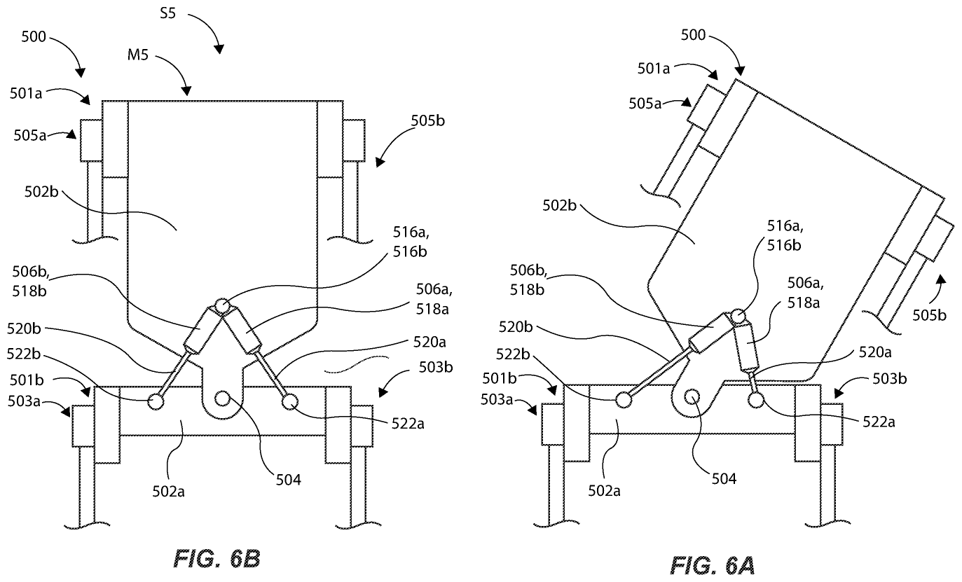

FIGS. 6A and 6B illustrate a robotic system comprising an upper robotic assembly and a base platform rotatably coupled together via one or more joints configured to facilitate movement of the upper robotic assembly relative to the lower robotic assembly in at least one degree of freedom. In the example shown, the robotic system is configured as a robotic exoskeleton 500, with the upper robotic assembly comprising an upper type of exoskeleton body section, and the base platform comprising a lower type of exoskeleton body section, wherein the upper and lower body exoskeleton sections together define the full body exoskeleton 500. The exoskeleton 500 can be operable to support itself in a stable support position, which in some examples, can comprise a self-standing, upright position (including the upper body exoskeleton section being supported in an upright, default support position relative to the lower body exoskeleton section). In this example, the stable support position comprises the stable support or stable default position S5 (FIG. 6B) that can be achieved by the below discussed joint position restoration assembly, such as when the exoskeleton 500 is powered down and/or not operated by a user, in accordance with an example of the present disclosure. More specifically, the robotic system in the form of the robotic exoskeleton 500 can comprise an upper body exoskeleton section 501a (or simply an upper body exoskeleton 501a) rotatably coupled to a lower body exoskeleton 501b (or simply a lower body exoskeleton 501b) via one or more joints (e.g., see joint 504), such that these are rotatable relative to one another in one or more degrees of freedom. The joint 504 can comprise an actuatable joint. The lower body exoskeleton 501b can comprise a first support member 502a, which can support left and right leg sections or members 503a and 503b (such as leg sections or members of any of the lower body exoskeletons discussed herein, or others). The upper body exoskeleton 501a can comprise a second support member 502b rotatably coupled to the first support member 502a about, and that defines, the joint 504 that moves within and that facilitates movement within at least one degree of freedom of the upper body exoskeleton 501a relative to the lower body exoskeleton 501b. In the case of the exoskeleton 500 (again, the upper robotic assemblies discussed herein can comprise a robotic exoskeleton, a humanoid robot, a teleoperated robot, a robotic anti or other robot or robotic assembly types), the joint 504 can move in and define one or more degrees of freedom corresponding to a degree of freedom of an element of a human body, such as lateral or side-to-side torso motion of the human body, and/or forward-to-back torso motion, and/or rotation torso motion in any combination, or all of these. In this regard, note that joint 504 in the drawings is a schematic representation of a joint that may rotate in one, two, or three degrees of freedom. Further note that more than one joint (i.e., a plurality of joints) may be incorporated to facilitate rotation of the upper body exoskeleton 501a relative to the lower body exoskeleton 501b, such as a joint situated above joint 504 and that is wholly supported by support member(s) of the upper body exoskeleton 501a for twisting rotation, for instance. As indicated above, in any robotic system, such as humanoid or others, the joint 504 does not necessarily need to define one or more degrees of freedom corresponding to one or more degrees of freedom of an element of the human body. Indeed, the joint 504 can define and facilitate movement of the upper and lower body exoskeletons 501a and 501b of the exoskeleton 500 relative to one another, whether these correspond to one or more degrees of freedom of an element of the human body or not. Again, this is true for all of the joints in this or any of the other examples discussed herein, whether the joints are part of a lower or upper body portion of a robotic system.

The second support member 502b can support a backpack or other structural support for supporting components that operate the exoskeleton, such as batteries, computers, controllers, etc. The second support member 502b can support left and right exoskeleton arm sections or members 505a and 505b. Note that the second support member 502b is shown as a single structural support device, but it may include a number of structural components coupled together to form a portion of an upper body exoskeleton.

The exoskeleton 500 can include mass properties M5 associated with each respective joint, which in this case is the mass properties of the components of the robotic assembly associated with joint 504, including the left and right exoskeleton arms 505a and 505b, that generates a gravity induced torque on the joint 504 that may tend to rotate the upper body exoskeleton 501a to the left or right side of a centered, default position. The exoskeleton 500 can comprise one or more joint position restoration assemblies, each having one or more springs, and each associated and operable with one or more joints. In this example, the exoskeleton 500 comprises a joint position restoration assembly comprising springs 506a and 506b (e.g., torso stabilizer springs) operably coupled to respective adjacent support members 502a and 502b operable with the joint 504 to apply a restoring torque to the joint 504 (e.g., which can be an active, actuatable robotic joint or a passive robotic joint). The joint position restoration assembly with its spring 506a and 506b can further comprise or be operable with at least one of a mechanical linkage (which in some examples can be adjustable), a spring stiffness of the springs 506a and 506b (which in some examples can be adjustable), an equilibrium position (i.e. zero force and/or torque position) (which in some cases can be adjustable), and any other characteristic or property that can be configured, and in some examples tuned to generate a suitable restoring torque versus joint position profile that corresponds to the mass properties M5 of the upper body exoskeleton 501 acting on or at the joint 504, and that provides a spring-based restoring torque that compensates for the gravity-induced load or torque at the joint 504, such that the joint position restoration assembly with its springs 506a and 506b are each operable to position (i.e., restore) and to support (i.e., maintain) the upper body exoskeleton 501a in a generally upright or vertical position (i.e., the stable default position S5) via the restoring torques applied to the joint 504 by the respective springs 506a and 506b. For instance, if the mass properties M5 of the upper body exoskeleton 501a of the exoskeleton 500 associated with the joint 504 would exert a gravity-induced torque at the joint 504 of 20 Nm/degrees in the position shown, then the joint position restoration assembly with its spring 506a (and also similarly spring 506b) can be configured, and in some examples tuned so as to generate a suitable restoring torque versus joint position profile sufficient to apply a restoring force or torque that counteracts and overcomes the 20 Nm/degrees of gravity-induced torque at the joint 504, so that the joint position restoration assembly with its springs 506a and 506b can position (i.e., restore) and to support (i.e., maintain) the joint 504 in an upright stable, default position (i.e., to prevent rotation of the joint in a rotational direction toward the ground).

The springs 506a and 506b can each comprise an elastic element and can comprise any type of spring discussed herein and identified above, such as a pneumatic spring mechanism or device, or a nitrogen spring mechanism or device, a coil spring, or any others apparent to those skilled in the art. The springs 506a and 506b can include mounting portions 516a and 516b, respectively, that mount or attach to the second support member 502b, and which can include a common mounting portion that fixedly attaches the springs 506a and 506b to each other (or about the same axis) and to the support member 402b. This helps to prevent rotation of the springs 506a and 506b while the second support member 502b moves or pivots about joint 504, such as during normal operation of the exoskeleton 500. The springs 506a and 506b can further include respective gas chambers 518a and 518b that house and support a compressible gas, and can include respective moveable pistons 520a and 520b (supporting cylinders (not shown)) moveable through the gas chambers 518a and 518b to facilitate compression and expansion of gas therein, in the traditional sense of a pneumatic spring, or such as with a (substantially constant) nitrogen cylinder spring device. The movable pistons 520a and 520b can be attached to the first support member 502a in a suitable manner.

The joint position restoration assembly, with its associated restoring torque versus joint position profile, can be configured, and in some examples tuned to allow the upper body 501a to rotate to the left and right about joint 504 when the exoskeleton 500 is worn and operated by a user, so that the springs 506a and 506b can compress to a certain degree and not substantially interfere with the intended use of the exoskeleton (and also act as a gravity compensation component, such as discussed above). Thus, during use of the exoskeleton 500 the second support member 502b may be rotated to the left or right about the joint 504, such as during a lifting task with one arm. If the second support member 502b rotates to the right, as shown in FIG. 6A, the first spring 506a will compress to a certain degree and will store energy, thereby compensating for gravity acting on the upper body 501a. Then, as the user rotates to the left (such as during picking up an item with a right arm), the first spring 506a will release energy, which assists with actuation and rotation of joint 504. In this way, a smaller actuator (e.g., a smaller electromagnetic motor) may be used to rotate the joint 504 than would otherwise be needed without incorporation of the springs 506a and 506b. Moreover, if the user steps out of the exoskeleton 500 and the upper body 501a happens to be leaning to the left or right (of the center, default position of FIG. 6B), the springs 506a and 506b can cooperate to restore or position and to support the upper body 501a in a generally upright, default support position, as shown in FIG. 6B, which is the stable support position S5. Thus, the springs 506a and 506b can each apply a restoring torque to the joint 504 to restore and support the position of the joint 504 and the second support member 502b, and to maintain this position.

Note that the lower body exoskeleton 501b may be configured like any of the examples discussed herein, or others. In one example, the lower and upper body exoskeletons, defining a full body exoskeleton, can be a full body “self-standing” exoskeleton having one or more joint position restoration assemblies having respective one or more springs and mechanical linkages (for lower body joints, and for the torso joint(s)) that cooperate together to position (i.e., restore) and to support (i.e., maintain) the exoskeleton in a stable support position, which in one example, can comprise a self-standing position (lower body), or the generally upright, default centered position (upper body) (these being the stable supports positions discussed herein). Thus, when a user desires to re-use or again don the full body self-standing exoskeleton 500, the user can merely “step into” the exoskeleton 500 with comfort and ease because the exoskeleton 500 is already in a standing position and an upright position. Thus, an external system, such as a hoist or hanger, is not needed or required to re-use (or don) the exoskeleton 500.

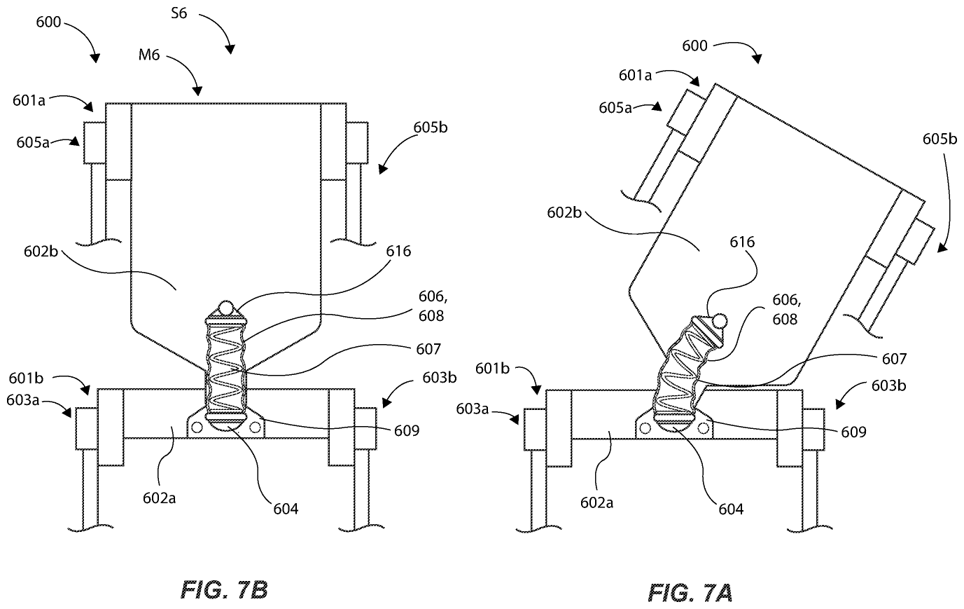

FIGS. 7A and 7B illustrate a robotic system comprising an upper robotic assembly and a base platform rotatably coupled together via one or more joints configured to facilitate movement of the upper robotic assembly relative to the lower robotic assembly in at least one degree of freedom. In the example shown, the robotic system is configured as a robotic exoskeleton 600, with the upper robotic assembly comprising an upper type of exoskeleton body section, and the base platform comprising a lower type of exoskeleton body section, wherein the upper and lower body exoskeleton sections together define the full body exoskeleton 600. The exoskeleton 600 is operable to achieve a stable support position, which in one example, can comprise the exoskeleton 600 being able to support itself in an upright position (including the upper body exoskeleton section being supported in an upright or centered, default position relative to the lower body exoskeleton section). In this example, the stable support position S6 (FIG. 7B) can be achieved by the below discussed joint position restoration assembly, such as when the exoskeleton 600 is powered down and/or not operated by a user, in accordance with an example of the present disclosure. More specifically, the exoskeleton 600 can comprise an upper body exoskeleton 601a (configured similarly as described above) pivotally or rotatably coupled to a lower body exoskeleton 601b by a spring 606, which defines a joint 604. The lower body exoskeleton 601b can comprise a first support member 602a, which can support left and right leg sections or members 603a and 603b (such as leg sections or members of any of the lower body exoskeletons discussed herein, or others). The upper body exoskeleton 601a can comprise a second support member 602b rotatably coupled to the first support member 602a via the joint 604, which support members are rotatable relative to one another via the spring 606 in this example. In the case of the exoskeleton 600 (again, the robotic assemblies discussed herein can comprise an exoskeleton, a humanoid robot, robotic arms, or other robot or robotic assembly types), the joint 604 can define a degree of freedom corresponding to one or more degrees of freedom of an element of a human body, such as lateral or side-to-side torso motion of the human body, and/or front-to-rear motion of the human body about the lower back or torso (an in between motions) and/or rotation about a vertical axis of the torso. In this regard, note that joint 604 in the drawings is a schematic representation of a joint that may rotate in one, two, or three degrees of freedom. The second support member 602b can support a backpack or other structural support for supporting components that facilitate operation of the exoskeleton, such as batteries, computers, controllers, etc. The second support member 602b can support left and right exoskeleton arm sections or members 605a and 605b. Note that the second support member 602b is shown as a single structural support device, but it may include a number of structural components coupled together to form a portion of an upper body exoskeleton.

The exoskeleton 600 can include mass properties M6 associated with the joint 604, and which can include the left and right exoskeleton arms 605a and 605b. The mass properties can generate a gravity induced torque on the joint 604 that may tend to rotate the upper body exoskeleton 601a to the left or right side of a centered, default position. The exoskeleton 600 can comprise one or more joint position restoration assemblies. In the example shown, the exoskeleton 600 can comprise a joint position restoration assembly comprising the spring 606 operably coupled to respective adjacent support members 602a and 602b and configured to apply a restoring torque to the joint 604 (which can be an actuated or passive joint), as defined by the spring 606, in one example. The spring 606 can have a spring stiffness value that can be configured, and in some examples tuned to generate a suitable restoring torque versus joint position profile that corresponds to the mass properties M6 of the upper body exoskeleton 601a acting on or at the joint 604, and that facilitates generation of a spring-based restoring torque by the joint position restoration assembly that compensates for the gravity-induced load or torque at the joint 604, such that the joint position restoration assembly with its spring 606 is operable to position (i.e., restore) and to support (i.e., maintain) the upper body exoskeleton 601a in a generally upright or vertical position (i.e., stable default position S6) via the restoring torque applied to the joint 604 by the spring 606. For instance, if the mass properties M6 of the upper body exoskeleton 601a of the exoskeleton 600 acting on or otherwise associated with the joint 604 would exert a gravity-induced torque on the joint 604 of 20 Nm/degrees in the position shown, then the joint position restoration assembly with its spring 606 can be configured, and in some examples tuned, so as to generate a suitable restoring torque versus joint position profile sufficient to facilitate generation and application of a restoring force or torque that counteracts and overcomes the 20 Nm/degrees gravity-induced torque at the joint 604 so that the joint position restoration assembly with its spring 606 can position (i.e., restore)) the joint 604 in an upright stable, default position, and to maintain it in this static or stable position (i.e., to prevent rotation of the joint in a direction away from the stable default position).

The spring 606 can comprise an elastic element configured to store and release energy, such as a coil spring 607 (or a different type of spring and elastic element, such as an elastomer, an elastomer spring, a polymer, a leaf spring, a helical spring, a negator spring, a constant force spring, a pneumatic spring, a rotary spring, a composite flexural spring, a torsion bar, or others as discussed herein and identified above or that would be apparent to those skilled in the art). The spring 606 can include a mounting portion 616 that mounts or attaches the spring 606 to the second support member 602b, and which can include a mount body and receive a fastener that fixedly attaches the spring 606 to the support member 602b. The spring 606 can further include a cover body 618 that surrounds or wraps around the coil spring 607, such as a flexible bellows or other flexible component. The other end of the spring 606 can be attached or mounted to the first support member 602a by suitable means, such as by a bracket 609 fastened to the first support member 602a that supports the end of the spring 606. Thus, the second support member 602b can bend or move about the spring 606, as illustrated in FIG. 7A. Alternatively, the coil spring 606 can be replaced with one or more leaf springs.

The joint position restoration assembly with its associated restoring torque versus joint position profile can be configured to allow the upper body 601a to rotate about the joint 604 when the exoskeleton 600 is worn and operated by a user, so that the spring 606 can compress or bend (i.e., flex) to a certain degree and not substantially interfere with the intended use of the exoskeleton (and also act as a gravity compensation component, such as discussed above). Thus, during use of the exoskeleton 600, and in one example, the second support member 602b may be rotated to the forward and to the right about the joint 604, such as during a lifting task with one arm. If the second support member 602b rotates to the right, as shown in FIG. 7A, the spring 606 will compress or bend (i.e., flex) to the right and forward to a certain degree, and will store energy, thereby compensating for gravity acting on the upper body 601a. Then, as the user rotates back and to the left (such as during picking up an item with a right arm), the spring 606 will release energy, which assists with actuation and rotation of joint 606. In this way, a smaller actuator (e.g., a smaller electromagnetic motor) may be used to rotate the joint 604 than would otherwise be needed without incorporation of the spring 606. Moreover, if the user steps out of the exoskeleton 600 and the upper body 601a happens to be leaning to the left or right of the center, default position shown in FIG. 7B, the spring 606 will operate to move and position and support the upper body 601a in a generally upright position, as shown in FIG. 7B. Thus, the spring 606 can apply a restoring torque to the joint 604 to position (i.e., restore) and maintain the position of the second support member 602b. Notably, the spring 606 is arranged in a central area relative to the axes of rotation of the joint 604, such that at least a portion of the spring 606 intersects one or more axes of rotation of the joint 604. In this way, the spring 606 is operable to bend or flex about three rotational degrees of freedom (xyz), which provides greater flexibility than with a system that only provides two rotational degrees of freedom. Thus, no matter the three dimensional position of the upper body exoskeleton relative to the lower body exoskeleton when the user steps out of the exoskeleton, the spring 606 operates to rotate the joint 604 to position the upper body exoskeleton in an equilibrium position, and generally vertically upright, as shown in FIG. 7B.

Note that the lower body exoskeleton 601b may be configured like any of the examples discussed herein, or others. In this manner, the lower and upper body exoskeletons, defining a full body robotic exoskeleton, can be a full body “self-standing” exoskeleton having a plurality of joint position restoration assemblies having respective one or more springs and linkages (for lower body joints, and for the torso joint(s)) that cooperate together to position (i.e., restore) and to support (i.e., maintain) the exoskeleton in a stable default position, such as a standing position (lower body), and the generally upright position (upper body). Thus, when a user desires to re-use and again don the full body self-standing exoskeleton 600, the user can merely “step into” the exoskeleton 600 with comfort and ease because the exoskeleton 600 is already in a standing position, and an upright position. Thus, an external system, such as a hoist or hanger, is not needed or required to support the exoskeleton 600 in such a position.

As indicated above, the particular robotic system configurations discussed above and shown in FIGS. 5A-7B are not meant to be limiting in any way. For example, although illustrating different lower body exoskeletons, the lower body exoskeletons 100, 200, 300 and 700 shown in FIGS. 1A-3C and 8A-8C, respectively, illustrate different examples and concepts of joint position restoration assemblies and how they are configured and implemented, as well as how they function or operate, with respect to the various joints and associated structural members making up the exoskeletons. Therefore, it is noted that the example configurations and functions of the joint position restoration assemblies as they are applied to the structural members and joints of the lower body exoskeletons 100, 200, 300 and 700 (namely the concepts of how various joint position restoration assemblies can be applied to be operable with various joints) can be applicable to a robotic system comprising an upper robotic system moveable relative to a base platform, as shown in FIGS. 4A-7B. For example, FIGS. 2A-2B illustrate how two joint position restoration assemblies comprising respective springs 206a and 206b can be operably coupled to respective support members 202a-d, as shown, where the first and second joint position restoration assemblies cooperatively operate to apply respective restoring torques to each of the three joints 204a-c to position and to support (i.e., maintain) the exoskeleton 200 in the stable support position. Indeed, two joint position restoration assemblies are operable to position and support three joints 204a-c in the stable support position by applying restorative torques to each of the joints 204a-c. This concept can be applied to a robotic system comprising an upper robotic assembly and a base platform that are moveable relative to one another via three joints (in series), where two joint position restoration assemblies can be operable to position and support the three joints in the stable support position by applying restorative torques to each of the three joints. To illustrate, the support member 202e and/or the torso structure 205 can be considered or thought of as an upper robotic assembly (or part of one), and the support member 202a can be thought of as a base platform, with the three support members 202b-d and the associated three joints 204a-c supporting these to be rotatable or otherwise moveable relative to one another. The two joint position restoration assemblies with their springs 206a and 206b can be configured to position and support the three joints 204a-c in the stable support position by applying restorative torques to each of the joints 204a-c, and thus position and support what can be considered the upper robotic assembly and the base platform in a stable support position relative to one another. The concepts shown in FIGS. 1A-1B, 3A-3C and 8A-8C and discussed above with respect to the respective joint position restoration assemblies and how they operate with the various joints and structural members in these examples can also be applied to a robotic system comprising an upper robotic assembly and a base platform moveable relative to one another as shown in FIGS. 4A-7B.

FIGS. 8A-C show a right leg exoskeleton 700 that is operable to achieve a stable support position S7, which in some examples, not being limited to this, can comprise a balanced, standing position (thus being a self-standing exoskeleton), when not in operation by a user, in accordance with an example of the present disclosure. Similarly as discussed above, the exoskeleton 700 can comprise a plurality of support members (e.g., see support members 702a-d) rotatably coupled together. Respective adjacent support members can be rotatably coupled together to form and define a plurality of joints (e.g., see joints 704a-c), each joint defining a degree of freedom. In the case of an exoskeleton, the degree of freedom can correspond to a degree of freedom of an element of the human body, such as an ankle (e.g., joint 704a), a knee (e.g., joint 704b), and a hip (e.g., joint 704c). Therefore, each joint 704a-c can operate about a respective axis of rotation 705a-c.

Note that the right leg exoskeleton 700 can be accompanied by a left leg exoskeleton that is a mirror copy of the right leg exoskeleton 700, and both can be operably coupled to a backpack or torso structure (e.g., 305) to for a lower body exoskeleton, and for supporting components that facilitate operation of the lower body exoskeleton. Further note that each joint 704a-c can be an actuator joint module having an electric motor, gear train(s), elastic component, sensor(s), etc., that make up a module or assembly for actuating the joint about the respective axis of rotation 705a-c. Thus, the support members 702a-d can each comprise a rigid housing that houses and supports components of each actuator joint module.

Each support member 702a-d can be rotatably coupled to at least one other or one additional support member to form and define a respective joint. In other words, adjacent support members from the various support members 702a-d can be coupled together to define a joint. For instance, support members 702a and 702b can be rotatably coupled together about and can form and define a first joint 704a, which provides the exoskeleton 700 with a degree of freedom corresponding to a degree of freedom of an ankle joint of a human leg for flexion/extension of the foot about the first joint 704a. Similarly, support members 702b and 702c can be rotatably coupled together about and can form and define a second joint 704b, which provides the exoskeleton 700 with a degree of freedom corresponding to a degree of freedom of a knee joint of the human leg for flexion/extension about the second joint 704b. And, support members 702c and 702d can be rotatably coupled together about and can form and define a third joint 704c, which provides the exoskeleton 700 with a degree of freedom corresponding to a degree of freedom of a hip joint of the human leg for flexion/extension about the third joint 704c. Finally, support member 702d can be rotatably coupled to another support member (e.g., 304e of FIG. 3A) to form and define a fourth joint (e.g., 304d of FIG. 3A).

The exoskeleton 700 can include mass properties M7 associated with each respective joint, which can include the mass properties of some or all of the components of the exoskeleton 700 (and other components not shown, such as the upper body exoskeleton components). As an overview, the exoskeleton 700 can include one or more joint position restoration assemblies, each comprising one or more springs (e.g., see spring 706), each one being operably coupled to one or more support members about one or more joints, such as to respective adjacent support members about a joint. In the example exoskeleton 700 shown, the spring 706 can operate to apply a restoring torque to respective joints 704b and 704c to position (i.e., restore) and to support (i.e., maintain) the exoskeleton 700 in the stable support position S7, which may be considered a restorative position of the exoskeleton 700. The joint position restoration assembly with its spring 706 can be configured to provide a suitable restoring torque versus joint position profile that corresponds to the mass properties M7 of the exoskeleton 700 (and others) associated with or otherwise acting on the joint 704b, such that the spring 706 is operable to position (i.e., restore) and to support (i.e., maintain) the exoskeleton 700 in the stable support position S7 via the restoring torques applied to the joint 704b by the spring 706. Regarding the configuration or tuning of the joint position restoration assembly with its spring 706, if the mass properties M7 of the portion of the exoskeleton 700 associated with the joint 704b are known, then each joint 704a-c may have a particular gravity-induced torque (at a particular rotational angle) that is experienced at each joint when the joints are not powered and when the exoskeleton 700 is not actuated by a user or operator, and is under the influence of gravitational forces and parasitic torque. That is, the joint torque includes gravity-induced torque of each joint along with inherent resistance of the joints (e.g., due to friction from bearings and gear train, cogging torque of an EM motor, and other such factors), which is the torque experienced at each particular joint due to gravitational forces exerted on the mass of one or more structural support members 702a-d, the torso structure, and other relevant masses of the exoskeleton 700 that may contribute to the mass properties M7. For instance, in one non-limiting example, assume a maximum gravity-induced torque of joint 704b (knee joint) is 20 Nm/degrees, which is calculated based on the mass properties of the components acting on or otherwise associated with the joint 704b. Based on this, the joint position restoration assembly with its spring 706 can be configured, and in some examples tuned, so as to provide a suitable restoring torque versus joint position profile (at this particular rotational position) sufficient to facilitate generation and application of a restoring force or torque that counteracts and overcomes the 20 Nm/degree gravity-induced torque at the joint 704b, so that the joint position restoration assembly with its spring 706 (and possible linkage devices) can support and restore to a restorative position and/or maintain in such position the joint 704b. The same principle applies to the configuration and/or tuning of a spring (not shown) that may be coupled to support members 702a and 702b to position (i.e., restore) and to support (i.e., maintain) the joint 704a via restoring torques applied by the spring to restrict or prohibit rotation of the joint 704a once supported in the stable support position S7 (see (e.g., 306a of FIG. 3A). In this manner, both springs (706 and another one like 306a) can cooperate together to support the exoskeleton 700 in the stable support position S7.

The spring 706 can comprise any type of spring as discussed herein as will be apparent to those skilled in the art. In one example, the spring 706 can comprise a linear spring in the form of a pneumatic spring, which includes a piston rod 709 translatable through a support housing 711 that contains a gas chamber, such as in the traditional configuration of a pneumatic compression spring. In another example, the spring 706 can comprise a linear spring in the form of a nitrogen cylinder spring device, which may provide the advantage of maintaining a substantially constant spring force during the stroke of the nitrogen cylinder spring device. The spring 706 can be mounted directly or indirectly to the second support body. In the example shown, the spring 706 can have one end 714a pivotally coupled to a spring support body 707a (as part of the support and restore to a restorative position and/or maintain in such position the joint) via a stator pin 708a (forming a linkage device including the spring and support member 702d), which facilitates rotation of the piston rod 709 relative to the support body 707a during operation. Note that FIG. 8C shows the stator pin 708a being exploded from the spring 706 and the support body 707a for purposes of illustration clarity. The spring support body 707a can be mounted (e.g., fastened) to the second support member 702b by suitable means, such as fasteners that extend through apertures 720 on a right side of the spring support body 707a to mount the support body 707a to the second support member 702b, and thereby mount the spring 706 to the second support member 702b. The spring support body 707a can comprise a pair of support flanges 715 spatially separated from each other and having a gap or opening that receives the end 714a of the piston rod 709. In this manner, the stator pin 708a can extend through the O-ring portion of the end 714a, and through opposing side apertures 713 formed through the support flanges 715 of the spring support body 707a to pivotally couple the spring 706 to the spring support body 707a and the second support member 702b (forming a linkage device between the spring and the support member 702c). Note that the spring support body 707a can have a plurality of sets of opposing side apertures 713 (or alternatively a slot) (i.e., mounting points) to provide a variable mounting position system, which is also part of the joint position restoration assembly (in other words the joint position restoration assembly comprises the variable mounting position system), to change the mounting location or position of the spring 706 as coupled to the second support member 702b. This can be useful for modifying or tuning and optimizing the spring stiffness and equilibrium position values of the spring 706 to achieve a desired restoring torque versus joint position profile without modifying the spring, or swapping out the spring itself for another spring. This can also be useful to accommodate different users having varying body heights, because a particular user may have a different anatomy, which may naturally position the knee joint 704b at a different position than the hip joint 704c. This matters because, when the user wearing and operating the exoskeleton, it is desirable for the spring 706 to not be too stiff or produce a restoring torque, which may unnecessarily impede rotation of the joints 704b and/or 704b, which would require a greater actuation force to actuate the joints 704b and/or 704c. Consequently, the electric motor(s) used to actuate the joint(s) may require a greater power requirement, which reduces efficiency of the system.

As shown in FIG. 8B, the other end 714b of the spring 706 can be pivotally coupled to another spring support body 707b mounted to the third support structure 702c via a stator pin 708b (similarly as the other end 714a of the spring 706) to pivotally couple the spring 706 to the third support structure 702c. In this way, the joint position restoration assembly with its spring 706 can apply a restoring torque to the joint 704b to rotate or actuate the joint 704b to the stable support position S7. Indeed, once the operator steps out of the exoskeleton 700, and the exoskeleton 700 is powered down so that its joint actuators are inactive, the spring 706 can release stored energy and expand to the position of FIG. 8A, thereby applying a restoring torque to support the joint 704b in the stable support position S7, and then to restrict or limit the rotation of joint 704b to prevent the support member 702d from collapsing under gravitational forces, thus maintaining the stable support position S7. Note that the spring support bodies 707a and 707b, and their pinned mountings to the spring, can each be considered “a linkage device” that couples together or links the spring to respective support members to facilitate the transfer of linear motion of the spring 706 to rotational motion to rotate the joint 704b accordingly. Such a linkage device can be configured, and in some examples tuned, in cooperation with the selection and configuration of the restoring spring to provide the desired restoring torque versus joint position profile.

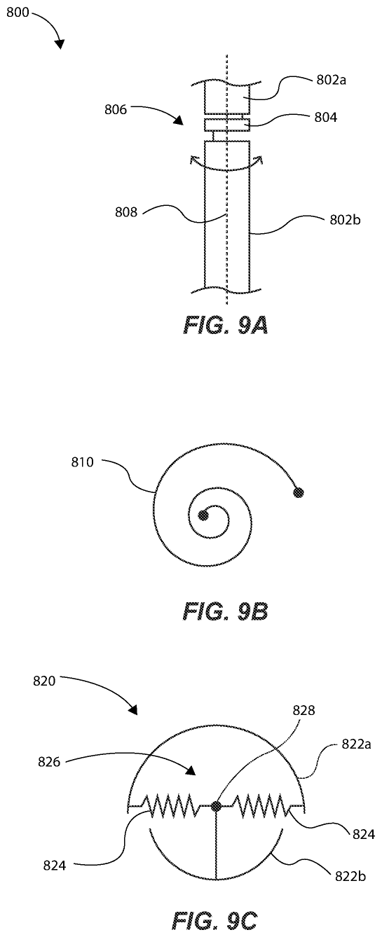

FIGS. 9A-9C illustrate examples of joint position restoration assemblies with respective spring configurations that are not necessarily associated with offsetting gravity-induced torque at a joint, but rather usable to return a joint to a stable support position with respect to medial/lateral rotation of the joint along a longitudinal axis, such as for medial/lateral rotation of a lower body exoskeleton corresponding to a medial/lateral degree of freedom of a human (e.g., femoral medial/lateral rotation), as further exemplified below. FIG. 9A schematically illustrates a robotic assembly 800 having a first support member 802a and a second support member 802b rotatably coupled together by a spring 804 to form and define a joint 806, such as for calf/ankle rotation (femoral rotation), or even for a torso twisting rotation, both in the case of an exoskeleton or humanoid type of robotic assembly. FIGS. 9B and 9C show top-down schematic views of example configurations of spring(s) that can be implemented in the robotic assembly 800 of FIG. 9A. Note that the joint 806 can comprise a joint module or mechanism that includes other components not shown, such as an actuator, clutch, transmission, gears, etc. for effectuating powered rotation of the joint 806 about axis 808. Further note that the spring 804 can comprise one or more springs, and can comprise any suitable spring. The examples of FIGS. 9A-9C can be implemented with the joint (femoral rotation) proximate support member 702b of FIGS. 8A and 8C, for instance.

More specifically, assume a torsional spring 810 (FIG. 9B) is used as the spring 804 (FIG. 9A), such that one end of the torsional spring 810 is coupled to the first support member 802a, and the other end of the torsional spring 810 is coupled to the second support member 802b. In this manner, the torsional spring 810 can be configured to position and maintain the joint 806 in a center or default position (e.g., stable support position) when the robotic assembly is not being operated or worn (or when worn but the user or actuator is not active to apply a force to rotate the joint 806). In this position, the torsional spring 810 is neither compressed nor expanded, rather it is in an equilibrium position that maintains proper alignment of the first and second support members relative to each other along the longitudinal axis 808. Accordingly, during use or rotation about the joint 806 (thereby moving the joint to an actuated position either clockwise or counter clockwise directions), the second support member 802b may rotate about axis 808 relative to the first support member 802a (e.g., corresponding to femoral rotation of a user, and/or active actuation), which thereby compresses (or expands) the torsional spring 810. Upon rotation from the stable support position to an actuated position, the spring 810 can apply a restoring torque to the joint 806 in the opposite direction of rotation of the joint 804. Accordingly, when the force is removed or reduced that caused the joint 806 to initially rotate/actuate (e.g., the user releases a force, and/or the actuator is unpowered or underpowered), the spring 810 releases its stored energy, which causes the joint 806 to rotate about axis 808 to return the joint 806 from the actuated position to the stable support position (e.g., see FIG. 9C). Note that the spring 810 may assist rotation by a powered actuator when the actuator is used to rotate the joint back to the stable support position, which thereby results in the actuator using less power to effectuate a rotation.

FIG. 9C is a top-down schematic view of a joint of a robotic assembly 820, such as the robotic assembly shown and described regarding FIG. 9A. In this example, a first support member 822a and a second support member 822b are coupled together by one or more springs 824 (e.g., leaf, coil, elastomer, etc.) to form and define a joint 826 that rotates about an axis of rotation 828 (into the page as viewed in FIG. 9C). In this example, the joint position restoration assembly with its one or more springs 824 are configured to position and maintain the joint 826 in a center or default position (e.g., stable support position) when the robotic assembly is not being operated or worn (or when worn but the user or actuator is not applying a force to rotate the joint 826). In this position, the one or more springs 824 are neither compressed nor expanded, rather they are in an equilibrium position that maintains proper alignment of the first and second support members relative to each other along the axis 828. Accordingly, the first support member 822a can rotate about axis 828 relative to the second support member 822b, which compresses or expands the one or more springs 824. Thus, the one or more springs 824 apply a restoring torque to the joint 826 when the joint 826 is rotated out of its stable support position and into an actuated position. When the force is removed or reduced that caused the joint 826 to rotate/actuate (e.g., the user releases a force, and/or the actuator is unpowered or underpowered), the one or more springs 824 release energy, which causes the joint 826 to rotate about axis 828 to return the joint 826 from the actuated position to the stable support position (the stable support position as shown in FIG. 9C). Note that the spring(s) 824 may assist rotation by a powered actuator when the actuator is used to rotate the joint back to the stable support position, which thereby results in the actuator using less power to effectuate a rotation.

The benefit of the joint position restoration assembly examples of FIGS. 9A-9C is that the center or default position is the preferable position for common activities like standing or walking on level ground, thereby reducing overall energy expenditure (i.e., by the user and/or a powered actuator) because the spring(s) help to maintain a center or default position without requiring actuation by an actuator to position the joint 826 in the stable support position. This can have additional safety benefits in the event that power is interrupted to one or more joints, because the joint (e.g., 806, 826) can maintain a desirable position if the user falls over, for instance.

FIG. 10A shows a side view of a joint of a robotic assembly 900 that includes a joint position restoration assembly comprising a spring 902 (i.e., pneumatic type of spring, or any other type as will be apparent to those skilled in the art) and a linkage device 904 that are configured to apply a restoring torque to a joint, which can be incorporated into any of the joint position restoration assembly examples discussed herein to provide additional mechanical advantage to any of the joints and joint position restoration assemblies discussed herein. More specifically, first and second support members 906a and 906b (shown schematically) can be rotatably coupled together about a joint 908 that defines an axis of rotation (e.g., a knee joint of an exoskeleton for flexion/extension). Note that, as part of the joint 908, a number of other components may be operatively coupled together to form a joint module or mechanism for actuating the joint 908 (e.g., actuator, clutch, transmission, etc.). One end of the spring 902 can be pivotally coupled to the first support member 906a, and the other end of the spring 902 can be pivotally coupled to a mounting portion 912 on the linkage device 904 (e.g., a simple crank). The linkage device 904 can be attached to the first and second support members 906a and 906b in a suitable manner to effectuate rotation of one support member relative to another (e.g., the clam shells of the linkage device 904 can be clamped or attached to the support members 906a and 906b by known means to translate linear motion from the spring to rotational motion about the joint), thus, along with the first and second support members 906a and 906b, forming a mechanical linkage about the joint 908. Similarly as discussed above regarding FIGS. 1A-8C, in this example the robotic assembly 900 (and other components not shown here) can include mass properties acting on or otherwise associated with joint 908, and the joint position restoration assembly with its spring 902 can operate to apply a restoring torque to joint 908 to position (i.e., restore) and to support (i.e., maintain) the joint 908 in the stable support position, which may be considered a restorative position. Accordingly, the joint position restoration assembly with its spring 902 and linkage device 904 is designed and arranged to apply a restoring torque that corresponds to the gravity-induced torque resulting from the mass properties acting on or otherwise associated with the joint to position and maintain the joint 908 in the stable support position. That is, the size, shape, type, spring stiffness, etc. can be selected and configured, along with the kinematics and arrangement of the linkage device 904, to produce the required restoring torque versus joint position profile and associated restoring torque to restore and support the joint 908 to/in the stable support position, as also discussed with similar examples herein.

FIG. 10B shows a side view of a joint of a robotic assembly 920 that includes a joint position restoration assembly comprising a spring 922 (i.e., a pneumatic type of spring, or any other type of spring as will be apparent to those skilled in the art) and a linkage device 924 that are configured to apply a restoring torque to a joint, which can be incorporated into any of the joint position restoration assembly examples discussed herein to provide additional mechanical advantage to any of the joints and joint position restoration assemblies discussed herein. More specifically, first and second support members 926a and 926b (shown schematically) can be rotatably coupled together about a joint 928 that defines an axis of rotation (e.g., a knee joint of an exoskeleton for flexion/extension). Note that, as part of the joint 928, a number of other components may be operatively coupled together to form a joint module or mechanism for actuating the joint (e.g., an actuator, a clutch, a transmission, etc.). One end of the spring 922 can be pivotally coupled to the first support member 926a, and the other end of the spring 922 can be pivotally coupled to the linkage device 924 (e.g., providing a four-bar linkage, such as a back-hoe linkage). The linkage device 924 can comprise three support links 934a, 934b, and 934c that are pivotally linked together and that couple together the first and second support members 926a and 926b to form a mechanical linkage. More specifically, the first support link 934a can be pivotally coupled to the first support member 926a at attachment portion 936 (e.g., by a pin). The first support link 934a can also be pivotally coupled to one end/rod of the spring 922 at attachment portion 938 proximate the middle section of the first support link 934a. At the other end, the first support link 934a is pivotally coupled to the second support link 934b, and the second support link 934b is pivotally coupled to the third support link 934c, which is attached or mounted to the second support member 926b. Accordingly, expansion or linear movement of the rod of the spring 922 exerts a force to the linkage device 924, which exerts a rotational force or torque between the support members 926a and 926b to rotate/actuate the joint 924 in the counter clockwise direction, for instance. Similarly as discussed above regarding FIGS. 1A-8C, in this example the robotic assembly 920 (and other components not shown here) include mass properties acting on or otherwise associated with the joint 928, and the joint position restoration assembly with its spring 922 and mechanical linkage 924 can operate to apply a restoring torque to the joint 928 to position i.e., restore) and to support (i.e., maintain) it in the stable support position, which may be considered a restorative position. Accordingly, the joint position restoration assembly with its spring 922 and linkage device 924 are designed and arranged to apply a restoring torque that compensates for and overcomes the gravity-induced torque resulting from the mass properties associated with the joint to restore and/or maintain the joint 908 in the stable support position. That is, the size, shape, type, spring stiffness, etc. can be selected and configured, along with the kinematics and arrangement of the linkage device 924, to produce the required restoring torque versus joint position profile and associated restoring torque to restore and support the joint 928 in/to the stable support position, as also discussed with similar examples herein.

It should be appreciated that various other linkage devices can be implemented in any of the joint position restoration assemblies discussed herein to suit a particular design to operate with a spring to apply a torque or force that restores or actuates a joint to a desired restorative rotational position (e.g., linkages that convert motion from linear to linear, rotary to linear, linear to rotary, and rotary to rotary, depending on the type of spring and linkage device implemented).

Both the examples of FIGS. 10A and 10B illustrate that, for a particular arrangement and joint, the spring and the linkage device can be collectively sized and configured to generate a desired restoring torque versus joint position profile, relative to the joint, that corresponds to the mass properties of at least a portion of the robotic assembly. That is, the joint position restoration assembly with its spring and all the design parameters for the joint position restoration assembly, can be configured, and in some examples tuned, based on, at least partially, the restoring torque required to actuate the particular joint to the stable support position throughout a range of rotational positions of the joint. In conjunction with the configuration of the spring, the particular mechanical linkage, with or without a linkage device, and its design parameters such as size, shape, mass, mounting location(s), kinetics, kinematics, etc., along with the resulting linkage, can be selected and configured, and in some examples tuned, based on the torque required to rotate and restore the particular joint to the stable support position throughout a range of rotational positions of the joint.

Note that the various rod ends and connection points of the springs, linkages, etc. discussed herein can comprise spherical rod ends or bearings that help facilitate slight rotation in additional degrees of freedom, which may be useful in cases where one spring can restore a joint(s) in multiple degrees of freedom.

It is noted that the specific types, placement and function of the springs discussed herein are not intended to be limiting in any way. Indeed, those skilled in the art will recognize that, for example, depending upon the type and configuration of a particular exoskeleton, that different types of springs, different number of springs, different relative placement of springs, and/or different sizes of springs can be selected to provide the most efficient and suitable arrangement about the joints of the exoskeleton to achieve restoring and/or supporting and maintaining the exoskeleton in a stable support position (e.g., an upright or standing position), such that the exoskeleton is self-supporting in these positions under the influence of gravitational forces.

It is further noted that, although the present disclosure focuses on exoskeletons, that the technology discussed herein is not intended to be limited to exoskeleton type robots or robotic assemblies. Indeed, those skilled in the art will recognize that other types of robots or robotic devices or robotic assemblies and systems (e.g., humanoid robots, teleoperated robots, robotic limbs (e.g., robotic arms), robotic end effectors, parallel mechanisms such as a Stewart platform, and others) can benefit from the technology discussed herein, namely to place one or more joints and the support members operating about and defining the one or more joints in a stable support position, which position does not necessarily need to be an upright or standing position. For example, a stable support position may include placing and supporting one or more jointed support members about any orientation. In addition, the stable support position can be subjected to forces in addition to or other than gravitational forces in which the stable support position is intended to overcome and in which the jointed support members are intended to be self-supporting with respect to. Therefore, an upright or standing stable support position is not intended to be limiting in any way.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the disclosure may not expressly disclose that some embodiments or features described herein may be combined with other embodiments or features described herein, this disclosure should be read to describe any such combinations that would be practicable by one of ordinary skill in the art. The use of “or” in this disclosure should be understood to mean non-exclusive or, i.e., “and/or,” unless otherwise indicated herein.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A robotic assembly, comprising:
a first joint comprising first and second support members rotatably coupled together; and
a joint position restoration assembly coupled to at least one of the first or second support members, and comprising a first spring, the joint position restoration assembly being operable to apply a restoring torque to the first joint,
wherein the joint position restoration assembly is configured to apply the restoring torque based on a restoring torque versus joint position profile relative to the first joint that corresponds to mass properties of at least a portion of the robotic assembly associated with the first joint, such that the joint position restoration assembly operates to apply the restoring torque to position and to support the first joint in a stable support position.

2. The robotic assembly of claim 1, wherein the joint position restoration assembly further comprises a mechanical linkage coupled between the first spring and at least one of the first or second support members, the mechanical linkage and the first spring operating together to facilitate application of the restoring torque.

3. The robotic assembly of claim 2, wherein the mechanical linkage further comprises a linkage device that operates with the spring to form, at least in part, the mechanical linkage.

4. The robotic assembly of claim 3, wherein one end of the first spring is coupled to the first support member and the other end of the first spring is coupled to the linkage device.

5. The robotic assembly of claim 2, wherein the mechanical linkage comprises at least one of a simple crank, a four-bar linkage, a planetary transmission, or a roller screw.

6. The robotic assembly of claim 2, wherein the first spring and the mechanical linkage are collectively configured to provide the restoring torque versus joint position profile and the resulting restoring torque, relative to the first joint.

7. The robotic assembly of claim 1, wherein the first spring comprises one of a pneumatic spring, a negator spring, a constant force spring, a linear spring, a rotary spring, a leaf spring, an elastomer spring, a composite flexural spring, a torsion bar, or a coil spring.

8. The robotic assembly of claim 1, wherein the first spring is continuously engaged to apply the restoring torque independent of the rotational position of the first joint.

9. The robotic assembly of claim 1, further comprising:
a second joint comprising the second support member and a third support member rotatably coupled to the second support member; and
a second joint position restoration assembly coupled to at least one of the second or third support members, and comprising a second spring, the second joint position restoration assembly being operable to apply a restoring torque to the second joint,
wherein the second joint position restoration assembly is configured to apply the restoring torque to the second joint based on a restoring torque versus joint position profile relative to the second joint that corresponds to known mass properties of at least a portion of the robotic assembly associated with the second joint, such that the second joint position restoration assembly operates to apply the restoring torque to position and to support the second joint in a stable support position.

10. The robotic assembly of claim 9, further comprising:
a third joint comprising the first support member and a fourth support member rotatably coupled to the first support member; and
a third joint position restoration assembly coupled to at least one of the first or fourth support members, and comprising a third spring, the third joint position restoration assembly being operable to apply a restoring torque to the third joint,
wherein the third joint position restoration assembly is configured to apply the restoring torque to the third joint based on a restoring torque versus joint position profile relative to the third joint that corresponds to mass properties of at least a portion of the robotic assembly associated with the third joint, such that the third joint position restoration assembly operates to apply the restoring torque to support the third joint in a stable support position.

11. The robotic assembly of claim 10, wherein each of the first, second and third joint position restoration assemblies is configured to correspond to known mass properties of the robotic assembly associated with the first, second and third joints, respectively, such that the first, second, and third joint position restoration assemblies are cooperatively operable to apply restoring torques to the first, second and third joints, respectively, to position and support the first, second, and third joints in respective stable support positions.

12. The robotic assembly of claim 1, wherein the joint position restoration assembly is coupled to the first and second support, members, the robotic assembly further comprising:
a second joint comprising the second support member and a third support member rotatably coupled to the second support member;
a third joint comprising the third support member and a fourth support member rotatably coupled to the third support member; and
a second joint position restoration assembly coupled to the second and the fourth support members, and comprising a second spring, the second joint position restoration assembly being operable to apply a restoring torque to the second and third joints,
wherein the joint position restoration assembly and the second joint position restoration assembly cooperatively operate to apply respective restoring torques to each of the first, second and third joints to position and to support the first, second and third joints in respective stable support positions.

13. The robotic assembly of claim 1, wherein the robotic assembly comprises one of an exoskeleton, a robotic limb, or a humanoid robot.

14. The robotic assembly of claim 1, further comprising a second joint comprising the second support member and a third support member rotatably coupled to the second support member, wherein the joint position restoration assembly is coupled to the first and third support members, the joint position restoration assembly being operable to apply a restoring torque to the first and second joints to position and to support the first and second joints in respective stable support positions.

15. The robotic assembly of claim 1, wherein the robotic assembly comprises a lower body exoskeleton comprising:
a plurality of lower body support members, including the first and second support members, each rotatably coupled to at least one other support member;
a plurality of joints defined by adjacently coupled lower body support members of the plurality of lower body support, members; and
a plurality of joint position restoration assemblies, each being operably coupled between at least two lower body support members defining at least one of the plurality of joints, such that the plurality of joint position restoration assemblies are cooperatively operable to position and support the plurality of joints in a stable support position.

16. The robotic assembly of claim 15, wherein the robotic assembly comprises a full body exoskeleton, the lower body exoskeleton being operable with an upper body exoskeleton, the full body exoskeleton comprising:
a torso joint coupling the lower body exoskeleton to the upper body exoskeleton; and
a joint position restoration assembly comprising a plurality of torso stabilizer springs coupled between the upper body exoskeleton and the lower body exoskeleton, and operable to position and support the upper body exoskeleton in a stable support position relative to the lower body exoskeleton.

17. The robotic assembly of claim 1, wherein the robotic assembly comprises an exoskeleton, and wherein the joint position restoration assembly is operable to limit rotation of the first joint to prevent the first and second support members from moving when the exoskeleton is not operated by a user.

18. The robotic assembly of claim 1, wherein the joint position restoration assembly further comprises a variable mounting position system that facilitates varying of a coupling location of the first spring to at least one of the first and second support members to vary the restoring torque provided by the joint position restoration assembly.

19. The robotic assembly of claim 1, wherein the robotic assembly comprises an exoskeleton wearable by a user, and wherein the first joint defines at least one degree of freedom corresponding to a degree of freedom of an element of a human body.

20. The robotic assembly of claim 19, wherein the exoskeleton further comprises:
a plurality of joints, including the first joint, wherein at least some of the plurality of joints defines at least one degree of freedom corresponding to a degree of freedom of an element of a human body;
a plurality of support members, including the first and second support members, wherein two or more of the plurality of support members are rotatably coupled together about respective joints of the plurality of joints; and
a plurality of joint position restoration assemblies, including the joint position restoration assembly, each comprising a spring and a mechanical linkage operable with the spring, and each configured to apply respective restoring torques, including the restoring torque, based on a restoring torque versus joint position profile relative to one or more joints of the plurality of joints that corresponds to known mass properties of at least a portion of the robotic assembly associated with the one or more joints, respectively,
wherein each of the plurality of joint position restoration assemblies is operable to apply the respective restoring torques to at least one of the one or more joints to position and support the one or more joints in respective stable support positions.

21. The robotic assembly of claim 19, further comprising a second joint defining a degree of freedom corresponding to a degree of freedom of an element of the human body, wherein the joint position restoration assembly is operable to apply collaborative restoring torques, including the restoring torque, to position and to support the first and second joints in respective stable support positions.

22. The robotic assembly of claim 19, further comprising second and third joints operable with the first joint, and defining respective degrees of freedom corresponding to degrees of freedom of an element of the human body, wherein the joint position restoration assembly is operable to apply collaborative restoring torques, including the restoring torque, to position and to support the first, second and third joints in respective stable support positions.

23. A robotic assembly, comprising:
a first joint comprising first and second support members rotatably coupled together;
a first joint position restoration assembly comprising a first spring, and operable to apply a restoring torque to the first joint;
a second joint comprising the second support member and a third support member rotatably coupled to the second support member;
a third joint comprising the first support member and a fourth support member rotatably coupled to the first support member; and
a second joint position restoration assembly coupled to the second and the fourth support members, and comprising a second spring, the second joint position restoration assembly being operable to apply a restoring torque to the second and third joints,
wherein the first and second joint position restoration assemblies cooperatively operate to apply respective restoring torques to each of the first, second and third joints to position and to support the first, second and third joints in respective stable support positions.

24. The exoskeleton of claim 23, wherein the first and second springs are operable to facilitate application of the respective restoring torques to restore the first, second and third joints to respective stable support positions, wherein the first and second springs are continuously engaged independent of the rotational positions of the first, second and third joints during operation of the exoskeleton.

25. The exoskeleton of claim 23, wherein each of the first and second joint position restoration assemblies is configured to correspond to known mass properties of the robotic assembly associated with the first, second and third joints, such that the first and second joint position restoration assemblies are cooperatively operable to apply restoring torques to the first, second and third joints, respectively, to position and support the first, second, and third joints in respective stable support positions.

26. The exoskeleton of claim 23, wherein at least one of the first or second joint position restoration assemblies further comprises a variable mounting position system that facilitates varying a coupling location of at least one of the first or second joint position restoration assemblies to vary the restorative and gravity compensation functions of the at least one of the first or second joint position restoration assemblies.

27. The exoskeleton of claim 23, wherein at least one of the first or second joint position restoration assemblies further comprises a mechanical linkage.

28. A method for configuring a robotic assembly, comprising:
configuring a first joint to comprise first and second support members rotatably coupled together; and
configuring a joint position restoration assembly to be operable with the first joint, and to apply a restoring torque based on a restoring torque versus joint position profile relative to the first joint that corresponds to known mass properties of at least a portion of the robotic assembly associated with the first joint, such that the joint position restoration assembly operates to apply the restoring torque to position and to support the first joint in a stable support position.

29. The method of claim 28, wherein configuring the joint position restoration assembly to be operable With the first joint comprises configuring the joint position restoration assembly to comprise a first spring and a mechanical linkage coupled between the first spring and at least one of the first or second support members, the mechanical linkage and the first spring operating together to facilitate application of the restoring torque to restore the first joint to the stable support position.

30. The method of claim 29, wherein configuring the joint position restoration assembly to be operable with the first joint comprises coupling a first end of the first spring to the first support member and coupling a second end of the first spring to the second support member.

31. The method of claim 29, wherein configuring the joint position restoration assembly to be operable with the first joint comprises coupling a first end of the first spring to the first support member and coupling a second end of the first spring to a linkage device of the mechanical linkage.

32. The method of claim 28, further comprising:
configuring a second joint to comprise the second support member and a third support member rotatably coupled to the second support member; and
configuring a second joint position restoration assembly to be operable with the second joint, and to apply a restoring torque to the second joint based on a restoring torque versus joint position profile relative to the second joint that corresponds to known mass properties of at least a portion of the robotic assembly associated with the second joint, such that the second joint position restoration assembly operates to apply the restoring torque to position and to support the second joint in a stable support position.

33. The method of claim 28, further comprising:
configuring a third joint to comprise the first support member and a fourth support member rotatably coupled to the first support member; and
configuring a third joint position restoration assembly to be operable with the third joint, and to apply a restoring torque to the third joint based on a restoring torque versus joint position profile relative to the third joint that corresponds to known mass properties of at least a portion of the robotic assembly associated with the third joint, such that the third joint position restoration assembly operates to apply the restoring torque to the third joint to position and to support the third joint in a stable support position.

34. The method of claim 28, further comprising:
configuring a second joint to comprise the second support member and a third support member rotatably coupled to the second support member;
configuring a third joint to comprise the third support member and a fourth support member rotatably coupled to the third support member; and
configuring a second joint position restoration assembly to be coupled to the second and the fourth support members, the second joint position restoration assembly being operable to apply a restoring torque to the second and third joints,
wherein the first and second joint position restoration assemblies cooperatively operate to apply respective restoring torques to each of the first, second and third joints to position and to support the first, second and third joints in respective stable support positions.

35. The method of claim 28, further comprising:
configuring a second joint to comprise the second support member and a third support member rotatably coupled to the second support member; and
configuring the joint position restoration assembly to be operable with the first and second joints, and to apply the restoring torque based on the restoring torque versus joint position profile to be relative to the first and second joints, such that the restoring torque versus joint position profile corresponds to known mass properties of at least a portion of the robotic assembly associated with the first and second joints, and such that the joint position restoration assembly operates to apply respective restoring torques to position and to support the first and second joints in a stable support position.

36. The method of claim 28, further comprising configuring the joint position restoration assembly to comprise a variable mounting position system that facilitates varying of a coupling location of the first spring to at least one of the first and second support members to vary the restoring torque provided by the joint position restoration assembly.

37. A robotic assembly, comprising:
a joint operable to undergo medial/lateral rotation between a stable support position and an actuated position;
first and second support members rotatably coupled together about the joint; and
a joint position restoration assembly comprising a spring, and coupled to at least one of the first or second support members, the joint position restoration assembly being operable to apply a restoring torque to the joint to return the joint from the actuated position to the stable support position.

38. The robotic assembly of claim 37, wherein the joint position restoration assembly is configured to apply the restoring torque to the joint to return the joint in the stable support position upon powered actuation of the joint ceasing.

39. The robotic assembly of claim 37, wherein the spring comprises a torsional spring.

40. The robotic assembly of claim 37, wherein the joint defines a degree of freedom that corresponds to a degree of freedom of medial/lateral rotation of a human leg.
